(19) United States
(12) Patent Application Publication
(10) Pub. No.: US 2024/0242347 A1
(43) Pub. Date: Jul. 18, 2024

(54) METHODS, SYSTEMS, AND PROGRAM PRODUCTS FOR DETECTING LESIONS IN PROSTATES USING MAGNETIC RESONANCE IMAGING (MRI) IMAGES

(71) Applicant: The Research Foundation for the State University of New York, Albany, NY (US)

(72) Inventors: Thomas Sanford, Syracuse, NY (US); Gennady Bratslavsky, Fayetteville, NY (US)

(21) Appl. No.: 18/562,027

(22) PCT Filed: May 26, 2022
(86) PCT No.: PCT/US2022/031069

§ 371 Date: Nov. 17, 2023

Related U.S. Application Data
(60) Provisional application No. 63/321,953, filed on Mar. 21, 2022.
(60) Provisional application No. 63/194,123, filed on May 27, 2021.

(51) Int. Cl. G06T 7/00; A61B 5/00; A61B 5/055; G01R 33/56; G06T 5/70; G06T 7/11; G06T 7/174; G16H 15/00; G16H 30/40; G16H 50/20

(52) U.S. Cl. CPC G06T 7/0012; A61B 5/055; A61B 5/4381; G01R 33/5608; G06T 5/70; G06T 7/11; G06T 7/174; G16H 15/00; G16H 30/40; G16H 50/20; G06T 2207/10088; G06T 2207/20021; G06T 2207/20084; G06T 2207/20212; G06T 2207/30081; G06T 2207/30096

(57) ABSTRACT

A system, method, and computer program product for detecting lesions on a multiparametric prostate using magnetic resonance imaging (MRI). A magnetic resonance imaging (MRI) device generated a plurality of MRI images of a prostate for a patient, and at least one computing device in operable communication with the MRI device segments a plurality of MRI images to define zones of anatomical data relating to the prostate. The plurality of MRI images are collapsed into a single, combined image that is rewindowed into zones of intensity thresholds, then divided into a plurality of distinct regions that are each associated a zone of the prostate, with each region concatenated to identify individual region image intensity values. Each region image intensity value for each of the plurality of regions is compared one or more image intensity thresholds. A lesion can be detected on a portion of the prostate based on the comparison.

### CROSS-REFERENCE TO RELATED APPLICATION

This application claims the benefit, pursuant to 35 U.S.C. § 119(e), of U.S. provisional application No. 63/194,123 filed on May 27, 2021, and U.S. provisional application No. 63/321,953 filed on Mar. 21, 2022, the contents of which are hereby incorporated herein by this reference.

### BACKGROUND OF THE INVENTION

The disclosure relates generally to magnetic resonance imaging (MRI) image processing, and more particularly, to methods, systems, and program products for manipulating MRI images to detect lesions in prostates and modify MRI images to improve visual-diagnostic review.

Currently, American Urologic Association guidelines states that magnetic resonance imaging (MRI) of the prostate can improve the diagnosis of prostate cancer but should only be utilized at centers of excellence due to poor performance at non-expert centers. This is an example of an area of healthcare in which creates substantial disparities in the availability of potentially life-saving technology. Even in centers of excellence where prostate imaging may improve outcomes for patients in aggregate, there is substantial disagreement among radiologists with regards to detection and grading of lesion suspicious for prostate cancer. Furthermore, prostate MRI interpretation can be very time-intensive, taking up to 45 minutes when performing segmentation tasks needed for some software (i.e. DynaCAD).

### BRIEF DESCRIPTION OF THE INVENTION

Briefly described, in one embodiment, the disclosure provides a method of detecting lesions on a multiparametric prostate using magnetic resonance imaging (MRI). The method includes segmenting a plurality of MRI images to define: a first zone of the prostate depicted in each of the plurality of images, and a second zone of the prostate depicted in each of the plurality of image, the second zone surrounding the first zone, wherein each of the first zone and the second zone including anatomical data relating to the prostate; collapsing the plurality of MRI images into a single, combined image; rewindowing of the single, combined image by: determining a first image intensity spectrum for the first zone of the prostate depicted in the single, combined image, determining a second image intensity spectrum for the second zone of the prostate depicted in the single, combined image, and defining: a first image intensity threshold for the first zone of the prostate based on the first image intensity spectrum, and a second image intensity threshold for the second zone of the prostate based on the second image intensity spectrum; denoising of the single, combined image by: dividing the single, combined image into a plurality of distinct regions, each of the plurality of regions associated with one of the first zone or the second zone of the prostate, concatenating each of the plurality of regions to identify individual region image intensity values, comparing the region image intensity value for each of the plurality of regions to the first image intensity threshold or the second image intensity threshold based upon the region's association with the first zone or the second zone of the prostate, and identifying at least one positive region of the plurality of regions where the region image intensity values for the at least one positive region is equal to or greater than the first image intensity threshold or the second image intensity threshold; and identifying a portion of the prostate including a detected lesion based on the identified at least one positive region of the plurality of regions, the identified portion of the prostate corresponding to: the first zone or the second zone, and an anatomic location of the prostate based on the anatomical data relating to the prostate.

In another embodiment, the disclosure provides a computer program product for detecting lesions on a multiparametric prostate using magnetic resonance imaging (MRI), including a non-transitory computer readable storage medium having program instructions stored therein. The program instructions executable by a processor to cause a computing device to: segment a plurality of MRI images to define: a first zone of the prostate depicted in each of the plurality of images, and a second zone of the prostate depicted in each of the plurality of image, the second zone surrounding the first zone, wherein each of the first zone and the second zone including anatomical data relating to the prostate; collapse the plurality of MRI images into a single, combined image; rewindow the single, combined image by: determining a first image intensity spectrum for the first zone of the prostate depicted in the single, combined image, determining a second image intensity spectrum for the second zone of the prostate depicted in the single, combined image, and defining: a first image intensity threshold for the first zone of the prostate based on the first image intensity spectrum, and a second image intensity threshold for the second zone of the prostate based on the second image intensity spectrum; denoise the single, combined image by: dividing the single, combined image into a plurality of distinct regions, each of the plurality of regions associated with one of the first zone or the second zone of the prostate, concatenating each of the plurality of regions to identify individual region image intensity values, comparing the region image intensity value for each of the plurality of regions to the first image intensity threshold or the second image intensity threshold based upon the region's association with the first zone or the second zone of the prostate, and identifying at least one positive region of the plurality of regions where the region image intensity values for the at least one positive region is equal to or greater than the first image intensity threshold or the second image intensity threshold; and identify a portion of the prostate including a detected lesion based on the identified at least one positive region of the plurality of regions, the identified portion of the prostate corresponding to: the first zone or the second zone, and an anatomic location of the prostate based on the anatomical data relating to the prostate.

In a further embodiment, the disclosure provides a system including a magnetic resonance imaging (MRI) device for generating a plurality of MRI images of a prostate for a patient; and at least one computing device in operable communication with the MRI device, the at least one computing device configured to detect lesions on the prostate of the patient by: segmenting a plurality of MRI images to define: a first zone of the prostate depicted in each of the plurality of images, and a second zone of the prostate depicted in each of the plurality of image, the second zone surrounding the first zone, wherein each of the first zone and the second zone including anatomical data relating to the prostate; collapsing the plurality of MRI images into a single, combined image; rewindowing of the single, combined image by: determining a first image intensity spectrum for the first zone of the prostate depicted in the single, combined image, determining a second image intensity spectrum for the second zone of the prostate depicted in the single, combined image, and defining: a first image intensity threshold for the first zone of the prostate based on the first image intensity spectrum, and a second image intensity threshold for the second zone of the prostate based on the second image intensity spectrum; denoising of the single, combined image by: dividing the single, combined image into a plurality of distinct regions, each of the plurality of regions associated with one of the first zone or the second zone of the prostate, concatenating each of the plurality of regions to identify individual region image intensity values, comparing the region image intensity value for each of the plurality of regions to the first image intensity threshold or the second image intensity threshold based upon the region's association with the first zone or the second zone of the prostate, and identifying at least one positive region of the plurality of regions where the region image intensity values for the at least one positive region is equal to or greater than the first image intensity threshold or the second image intensity threshold; and identifying a portion of the prostate including a detected lesion based on the identified at least one positive region of the plurality of regions, the identified portion of the prostate corresponding to: the first zone or the second zone, and an anatomic location of the prostate based on the anatomical data relating to the prostate.

A further embodiment of the disclosure provides a method of detecting lesions on a multiparametric prostate using magnetic resonance imaging (MRI). The method including: segmenting a plurality of MRI images to define a plurality of zones, the plurality of zones including: a first zone of the prostate depicted in each of the plurality of images, and a second zone of the prostate depicted in each of the plurality of image, the second zone distinct from the first zone, wherein each of the first zone and the second zone including anatomical data relating to the prostate; collapsing the plurality of MRI images into a single, combined image; rewindowing of the single, combined image by: determining a first image intensity spectrum for the first zone of the prostate depicted in the single, combined image, determining a second image intensity spectrum for the second zone of the prostate depicted in the single, combined image, and defining: a first image intensity threshold for the first zone of the prostate based on the first image intensity spectrum, and a second image intensity threshold for the second zone of the prostate based on the second image intensity spectrum; denoising of the single, combined image by: dividing the single, combined image into a plurality of distinct regions, each of the plurality of regions associated with one of the first zone or the second zone of the prostate, concatenating each of the plurality of regions to identify individual region image intensity values, comparing the region image intensity value for each of the plurality of regions to the first image intensity threshold or the second image intensity threshold based upon the region's association with the first zone or the second zone of the prostate, and identifying at least one positive region of the plurality of regions where the region image intensity values for the at least one positive region is equal to or greater than the first image intensity threshold or the second image intensity threshold; and identifying a portion of the prostate including a detected lesion based on the identified at least one positive region of the plurality of regions, the identified portion of the prostate corresponding to: the first zone or the second zone, and an anatomic location of the prostate based on the anatomical data relating to the prostate.

The illustrative aspects of the present disclosure are designed to solve the problems herein described and/or other problems not discussed.

### BRIEF DESCRIPTION OF THE DRAWINGS

These and other features of this disclosure will be more readily understood from the following detailed description of the various aspects of the disclosure taken in conjunction with the accompanying drawings that depict various embodiments of the disclosure, in which:

FIG. 1 shows a schematic view of a system for detecting lesions on a prostate of a patient, according to embodiments of the disclosure.

FIGS. 2A-2C show various magnetic resonance imaging (MRI) images including distinct properties, according to embodiments of the disclosure.

FIG. 3A shows a computer-generated model of a patient's prostate including a plurality of mapped/identified regions or zones, according to embodiments of the disclosure.

FIG. 3B shows the plurality of identified zones on an MRI image of the patient's prostate, according to embodiments of the disclosure.

FIG. 4 shows a single, combined image formed by collapsing the plurality of MRI images of FIGS. 2A-2C, according to embodiments of the disclosure.

FIGS. 5A-5C show various views of the collapsed, single, combined image of FIG. 4 undergoing a rewindowing process, according to embodiments of the disclosure.

FIG. 6 shows a histogram graph representing an image intensity for a portion of the collapsed, single, combined image of FIG. 4, according to embodiments of the disclosure.

FIG. 7 shows a denoised version of the rewindowed, single, combined image of FIG. 5C, according to embodiments of the disclosure.

FIGS. 8 and 9 show various views of MRI images identifying a detected lesion within a patient's prostate, according to embodiments of the disclosure.

FIG. 10 shows a report including data relating to the detected lesion and a visual of where the detected lesion is located on the patient's prostate, according to embodiments of the disclosure.

FIGS. 11-18 show magnetic resonance imaging (MRI) images including distinct, defined zones, according to various embodiments of the disclosure.

FIG. 19 shows a denoised version of a rewindowed, single, combined MRI image, according to embodiments of the disclosure.

FIGS. 20-22 show various flowcharts illustrating processes for detecting lesions on a prostate of a patient, according to embodiments of the disclosure.

FIG. 23 shows a schematic view of a computing system configured to process MRI image(s) and detect lesions on a prostate of a patient, according to embodiments of the disclosure.

FIG. 24 shows a flowchart illustrating processes for corresponding a plurality of radiology images, according to embodiments of the disclosure.

FIG. 25 shows adjusted, sliced images of a plurality of radiology images of a patient after corresponding each of the plurality of radiology images, according to embodiments of the disclosure.

It is noted that the drawings of the disclosure are not to scale. The drawings are intended to depict only typical aspects of the disclosure, and therefore should not be considered as limiting the scope of the disclosure. In the drawings, like numbering represents like elements between the drawings.

### DETAILED DESCRIPTION OF THE INVENTION

As an initial matter, in order to clearly describe the current disclosure it will become necessary to select certain terminology when referring to and describing relevant components within the disclosure. When doing this, if possible, common industry terminology will be used and employed in a manner consistent with its accepted meaning. Unless otherwise stated, such terminology should be given a broad interpretation consistent with the context of the present application and the scope of the appended claims. Those of ordinary skill in the art will appreciate that often a particular component may be referred to using several different or overlapping terms. What may be described herein as being a single part may include and be referenced in another context as consisting of multiple components. Alternatively, what may be described herein as including multiple components may be referred to elsewhere as a single part.

As discussed herein, the disclosure relates generally to magnetic resonance imaging (MRI) image processing, and more particularly, to methods, systems, and program products for manipulating MRI images to detect lesions in prostates and modify MRI images to improve visual-diagnostic processes.

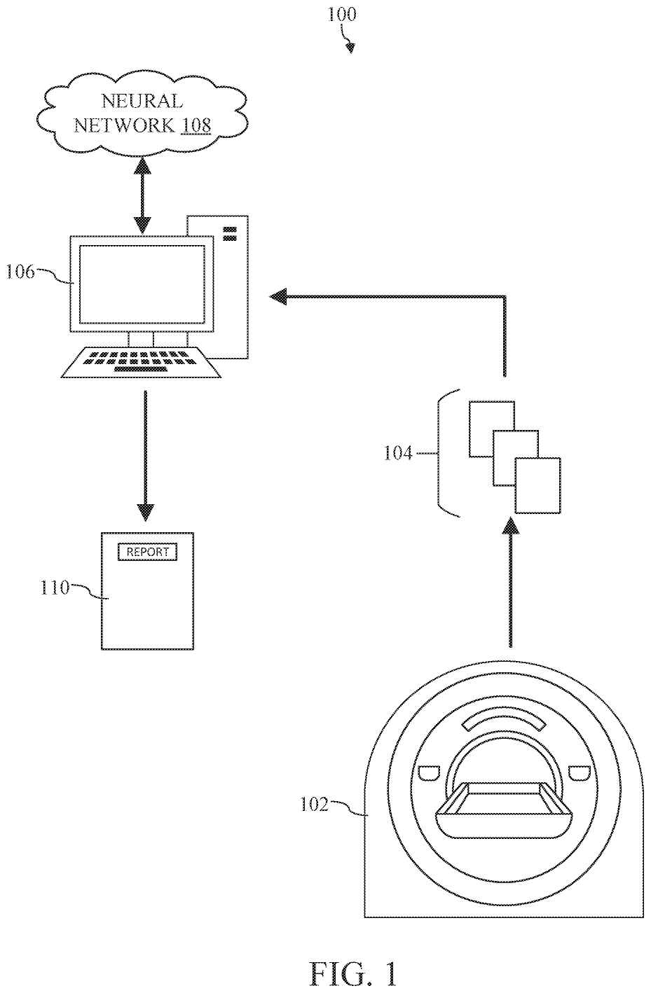

These and other embodiments are discussed below with reference to FIGS. 1-25. However, those skilled in the art will readily appreciate that the detailed description given herein with respect to these Figures is for explanatory purposes only and should not be construed as limiting.

FIG. 1 Shows a schematic view have a system 100 for processing magnetic resonance imaging (MRI) images. More specifically, FIG. 1 shows a non-limiting example of system 100 that may detect lesions on a multiparametric prostate using MRI and/or MRI images. System 100 may include an MRI Device 102 configured to generate MRI images 104 of a patient's prostate. MRI device 102 may be formed as any suitable magnetic resonance imaging device, apparatus, and/or system that may generate, create, form, and/or capture the desired MRI images 104 of an imaged prostate, as discussed herein.

MRI images 104 generated and/or captured by MRI device 102 may include various images having distinct imaging characteristics and/or properties. That is, MRI device 102 make capture a plurality of MRI images 104, where each of the plurality of MRI images 104 include distinct imaging characteristics and/or properties to aid in the detection of lesion(s) in an observed patient's prostate, as discussed herein. For example, the plurality of MRI images 104 generated/captured by MRI device 102 may include, but is not limited to, a low intensity MRI-image ranging from 0.7 Tesla (T)-1.2 T, a mid-intensity MRI-image ranging from 1.3 T-1.9 T, and/or a high-intensity MRI-image ranging from 2.0 T-3.0 T. In other non-limiting examples, the plurality of MRI images 104 may include, but is not limited to, hydrogen ion response to various strengths and intervals of radiofrequency signals (T1/T2), as well as, diffusion weighted imaging (DWI) or MR spectroscopy. Additionally, MRI images generated/captured by MRI device 102 and/or transmitted to distinct portions of system 100 (e.g., computing device 106) for processing may be, for example, a DICOM format. Although three MRI images are shown in FIG. 1, it is understood that MRI device 102 may generate/capture more or less MRI images 104. As discussed herein, processes for detecting lesions in patient's prostates may be performed using one or more MRI images 104.

As shown in FIG. 1, system 100 may also include at least one computing device 106. Computing device 106 may be in operable communication with MRI device 102. More specifically, computing device 106 may be connected to, in communication with, and/or operably connected with MRI device 102. As a result, and during operation, computing device 106 may receive MRI images 104 generated/captured by MRI device 102 and may perform processes an MRI images 104 to detect lesions within a patient's prostate as discussed herein. Computing device 106 may be a stand-alone device, or alternatively may be a portion and/or included in a larger computing device (not shown) of system 100. For example, and as shown in FIG. 1, computing device 106 may be separate from MRI device 102. Alternatively, computing device 106 may be part of the overall computing system that is used in the operation of MRI device 102. As such, computing device 106 may be formed as any device and/or computing system/network that may be configured to perform the processes discussed herein to identify or detect lesion(s) in a patient's imaged prostate. As discussed herein, computing device 106 may be configured to process MRI images 104 to detect lesion(s) in an imaged prostate. As shown in FIG. 1, computing device 106 may be in electronic communication with and/or communicatively coupled to various devices, apparatuses, and/or portions of system 100. In non-limiting examples, computing device 106 be hard-wired and/or wirelessly connected to and/or in communication with MRI device 102, and/or other components via any suitable electronic and/or mechanical communication component or technique. For example, computing device 106 may be in electronic communication with MRI device 102 and neural network 108. Additionally, and as discussed herein, computing device 106 may also receive, process, and/or analyze MRI images 104 during the processes discussed herein

System 100 may also include a neural network 108. In the non-limiting example shown in FIG. 1, neural network 108 may be distinct/separate from and in communication and/or operably coupled to computing device 106. In another non-limiting example, neural network 108 may be included within and/or formed as a part of computing device 106. Neural network 108 may be any suitable component, device, program product, and/or system that may be configured to aid in the process of detecting lesions within a patient's prostate based on MRI images 104, as discussed herein. For example, neural network 108 may be formed as any suitable machine learning device, program, and/or series of algorithms including, but not limited to, artificial/simulated neural networks including a plurality of interconnected, hidden layer nodes.

Upon receiving the plurality of MRI images 104 generated/captured by MRI device 102, computing device 106 using neural network 108 may perform a plurality of processes, manipulations and/or calculations using MRI images 104 to detect lesions within the imaged patient's prostate. Additionally, computing device 106 may generate a report 110 related to the findings, analysis, and/or potential detection of lesion(s). In non-limiting examples, report 110 may be a physical print out, a graphical depiction provided on a display device of computing device 106 (e.g., screen monitor), or any other suitable visual representation providing information or data relating to the analysis of MRI images 104 and/or the detection of lesion(s) in the image of patient's prostate. As discussed herein, the generated report 110 may include visual data relating to the identified portion of the prostate including detected lesion(s), probabilities for respective risk categories associated with the detected lesion(s), and/or options for modification for the report. Additionally, or alternatively, the generated report 110 may include a set of anatomic coordinates corresponding to the location of the detected lesion in the prostate, the likelihood the lesions contain clinically significant prostate cancer. It may also delineate the laterality and location of all detected lesions within prostate zonal anatomy and relevant sector of the prostate.

As discussed herein, report 110 generated by the processes performed by computing device 106 and neural network 108 may aid in a radiologists' and/or overseeing physicians' ability to detect lesion(s) in the prostate more quickly, and may auto generate additional inquiries and/or procedures to be performed in view of the detection of a lesion. Additionally, and as discussed herein, system 100 and the processes performed therein may provide radiologists and/or physicians the ability to detect lesions in an imaged patient's prostate quickly (e.g., in less than a minute) with improved accuracy (e.g., over 90% accuracy) and automation in detection over conventional processes/detection methods.

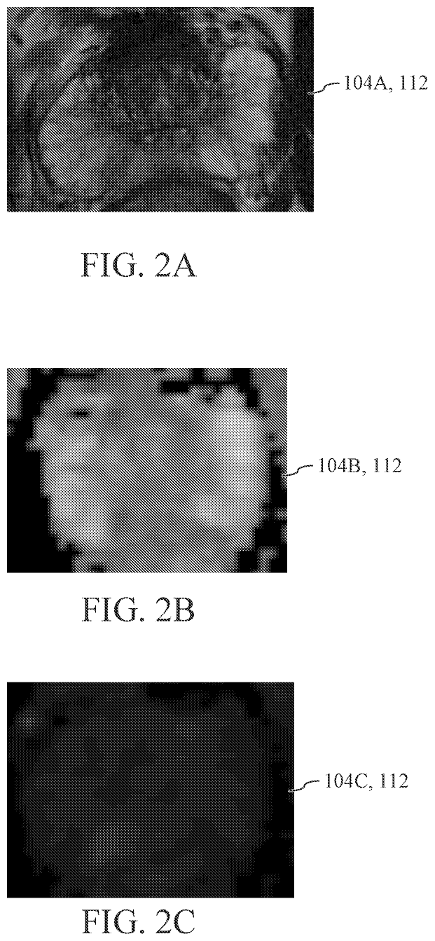

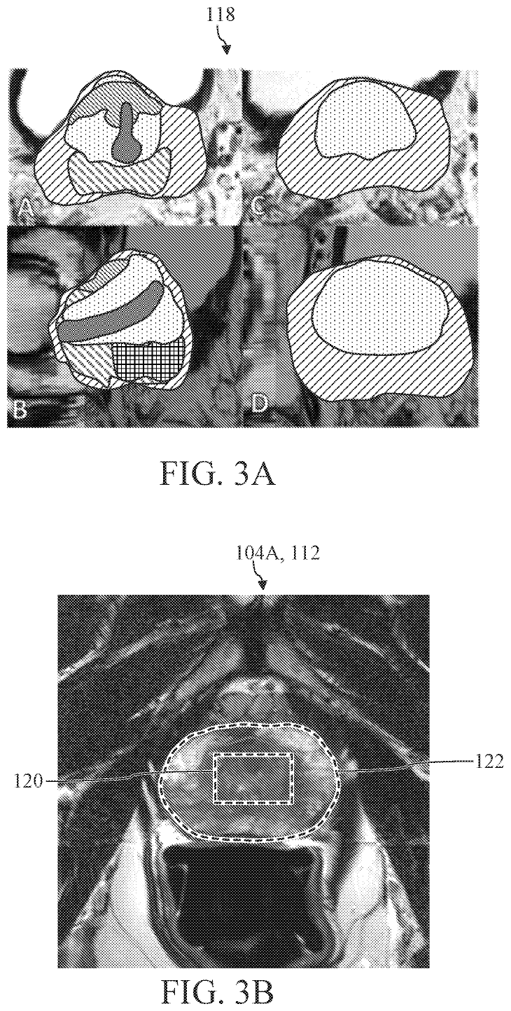

Turning to FIGS. 2A-10, and with continued reference to FIG. 1, processes for detecting lesion(s) on an imaged patient's prostate may be discussed herein. The processes discussed herein with respect to FIGS. 2A-10 may be performed by system 100 and the various components, devices, and/or systems included therein and shown and FIG. 1. It is understood that similarly numbered and/or named components may function in a substantially similar fashion. Redundant explanation of these components has been omitted for clarity.

FIGS. 2A-2C depict a plurality of MRI images 104. More specifically, MRI images 104A, 104B, 104C may be generated/captured by MRI device 102 of system 100 (FIG. 1). By comparison, MRI images 104A, 104B, 104C are distinct from one another and/or include distinct image characteristics/properties. For example, each of the plurality of MRI images 104A, 104B, 104C may include similar/constant field strength, but may be captured with distinct radiofrequency signal, which in turn may perturb hydrogen ions in each image 104A, 104B, 104C. In addition, or alternatively, each image 104A, 104B, 104C may include distinct gradients. In another non-limiting example, MRI image 104A may include a high intensity MRI image ranging from 2.0 T-3.0 T, MRI image 104B may include a mid-intensity MRI image ranging from 1.3 T-1.9 T, and MRI image 104C may include a low intensity MRI image ranging from 0.7 T-1.2 T. Each of the plurality of MRI images 104A. 104B, 104C may include anatomical data 112 relating to the image prostate. In non-limiting examples, anatomical data 112 may include the position and/or the orientation of the prostate/patient while being imaged by MRI device 102, as well as, anatomical direction and/or orientation of the prostate itself (e.g., anterior, posterior, right, left, etc.). Furthermore, each of the plurality of MRI images 104A, 104B, 104C may include additional data relevant to the processing of the images for detecting lesion(s) within the imaged prostate. For example, MRI images 104A, 104B, 104C may include T2 weighted images, Diffusion weighted images such usually b value >1400 s/mm2/Apparent Diffusion Coefficient map images, Dynamic Contrast Enhanced Images, and so on

FIG. 3A shows a computer-generated model 118 of a patient's prostate based on at least one of the plurality of MRI images 104A, 104B, 104C. As shown, computer generated model 118 may include a plurality of (computer generated) images of the imaged prostate in different orientations. Furthermore, and as shown in the non-limiting example of FIG. 3A, computer generated model 118 may also include, define, identify, and/or map a plurality of distinct regions and/or zones of the imaged prostate. Computer generated model 118 may be created, formed, and/or established using computing device 106 and/or neural network 108 of system 100. For example, computing device 106/neural network 108 of system 100 may use open source software Monai for generating model 118, and in turn defining, identifying, and/or mapping the plurality of distinct regions and/or zones in model 118.

The identified and/or mapped plurality of distinct regions and/or zones of the imaged prostate shown in computer generated model 118 of FIG. 3A may be the result of performing a segmentation process on model 118 and/or the plurality of MRI images 104. That is, computing device 106 and/or neural network 108 after receiving the plurality of MRI images 104 may perform a segmentation process to identify, map, and/or define a plurality of distinct zones of the imaged prostate. The segmentation process may be any suitable process that may partition each of the plurality of MRI images 104 into a plurality of segments in order to identify or define the plurality of zones, as discussed herein. In the non-limiting example shown in FIG. 3A, each of the plurality of distinct zones 120, 122 may be indicated, defined, and/or identified based on distinct colors, shading, or color gradient.

Once identified, mapped, and/or defined in computer generated model 118, computing device 106 may define/identify the plurality of distinct zones 120, 122 on at least one of the plurality of MRI images 104A, 104B, 104C based on or using the regions/zones mapped in model 118. That is, and turning to FIG. 3B, MRI image 104A, as previously shown in FIG. 2A, may include the identified, defined, and/or mapped zones 120, 122 defined during the segmentation process. The plurality of distinct zones 120, 122 may be overlaid, masked, included within, or otherwise depicted in each of the plurality of MRI images 104A, 104B, 104C using any suitable image editing and/or manipulation technique. The plurality of zones 120, 122 defined during the segmentation process may include at least a first zone 120 and a second zone 122, where second zone 122 is distinct from first zone 120. In the non-limiting example shown in FIG. 3B, first zone 120 may correspond to an inner zone of the prostate depicted in each of the plurality of images 104 (only 104A shown), and second zone 122 may correspond to an outer zone of the prostate depicted in each of the plurality of images 104 (only image 104A shown). In this example, the second/outer zone 122 may substantially surround the first/inner zone 120. In other non-limiting examples discussed herein (see, FIGS. 11-18), first zone 120 and second zone 122 may correspond or defined distinct portions/zones of the imaged prostate. Based on the anatomical data collected and/or determined using the plurality of images 104A, 104B, 104C generated by MRI device 102, the plurality of images 104A, 104B, 104C including the defined plurality of zones 120, 122 may also include anatomical data relating to the image prostate.

Although discussed herein as being (automatically) defined and/or identified using computing device 106/neural network 108 (e.g., Monai software), the segmentation process may also include adjusting first zone 120 and/or second zone 122. More specifically, the identified/defined first zone 120 and/or second zone 122 of the imaged prostate may be subsequently adjusted based on user input. In a non-limiting example, a user (e.g., radiologist, physician, etc.) may adjust the size, shape, and/or characteristics of first zone 120 and/or second zone 122 included in the plurality of images 104A, 104B, 104C based on, for example, associated anatomical data for the imaged prostate, preexisting imaging/medical information or expertise, and/or to aid in subsequent processes performed on the plurality of MRI images 104A, 104B, 104C discussed herein.

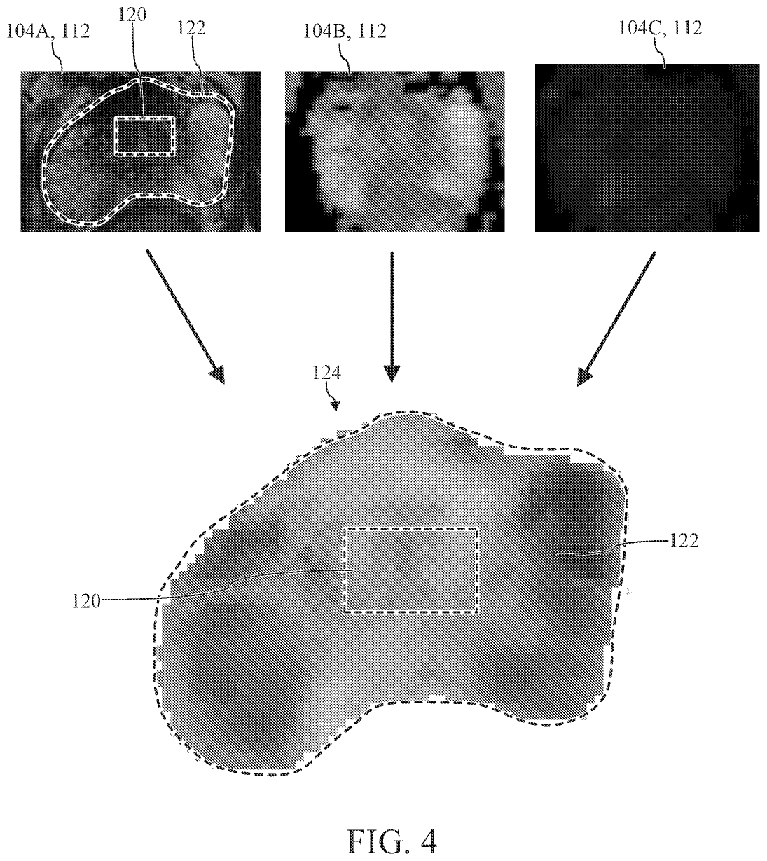

Turning to FIG. 4, a single, combined image 124 is shown. Specifically, FIG. 4 depicts a single, combined image 124 that may be formed by collapsing the plurality of MRI images 104A, 104B, 104C captured/generated by MRI device 102 of system 100 (see, FIG. 1). Collapsing the plurality of MRI images 104A, 104B, 104C to form single, combined image 124 may include first aligning each of the plurality of MRI images 104A, 104B, 104C based on, at least in part, the defined or identified plurality of zones (e.g., first zone 120, second zone 122) and/or the anatomical data relating to the imaged prostate and associated with each of the plurality of MRI images 104A, 104B, 104C. In a non-limiting example, collapsing the plurality of MRI images 104A, 104B, 104C may also include normalizing each of the aligned, plurality of MRI images 104A, 104B, 104C, and subsequently inverting a high-b value for each of the aligned, plurality of MRI images 104A, 104B, 104C. Following the inversion of the high-b value for each of the aligned, plurality of MRI images 104A, 104B, 104, collapsing the plurality of MRI images 104A, 104B, 104C may include segmenting each of the plurality of aligned, plurality of MRI images 104A, 104B, 104C, and subsequently inverting each of the segmented and aligned, plurality of MRI images 104A, 104B, 104C. The collapsing, and more specifically the various steps performed to collapse the plurality of MRI images 104A, 104B, 104C (e.g., aligning, normalizing, inverting, segmenting, inverting, etc.) may be performed by computing device 106 and/or neural network 108 of system 100 using any suitable components, device, programs, and/or systems.

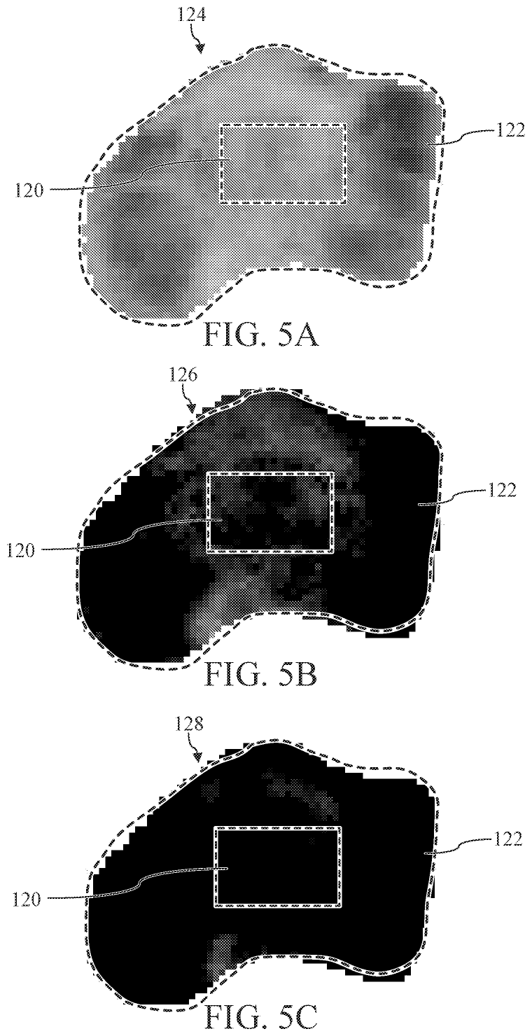

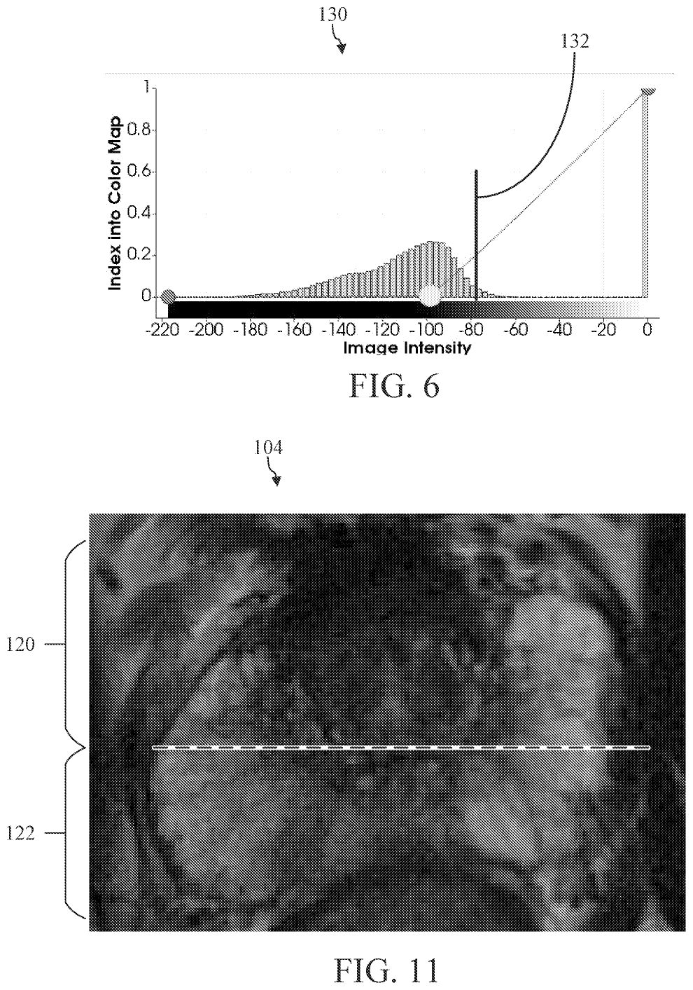

FIGS. 5A-6 show additional process performed by system 100 (see, FIG. 1) for detecting lesion(s) in an imaged patient's prostate using a plurality of MRI images 104. In the non-limiting example, newly formed single, combined image 124 may undergo a rewindowing process. That is, subsequent to collapsing the plurality of MRI images 104A, 104B, 104 to form single, combined image 124, single, combined image 124, as shown in FIG. 5A, may undergo a rewindowing process, a grey-level mapping process, a contrast stretching process, a histogram modification process, and/or contrast enhancement to manipulate the greyscale component of combined image 124. In non-limiting examples, the rewindowing process may include determining a plurality of image intensity spectrums. That is, rewindowing single, combined image 124 may include determining a first image intensity spectrum for first zone 120 (e.g., inner zone) of the imaged prostate depicted in single, combined image 124, and determining a second image intensity spectrum for second zone 122 (e.g., outer zone) of the imaged prostate. The determined image intensity spectrum may include, for example, a set of values based on the range of image intensity, pixel intensity, and/or an intensity value for each pixel based on gray-level image of single, combined image 124. As discussed herein, an image intensity spectrum may be determined for each of the plurality of zones 120, 122. In other non-limiting examples (see, FIG. 21), a single image intensity may be determined over the entirety of single, combined image 124. Additionally, the rewindowing of single, combined image 124 may also include defining a first image intensity threshold for first zone 120 (e.g., inner zone) of the imaged prostate based on the first image intensity spectrum and defining a second image intensity threshold for second zone 122 (e.g., outer zone) of the imaged prostate based on the second image intensity spectrum. The first image intensity threshold and the second image intensity threshold may be determined, calculated, and/or defined using computing device 106 and/or neural network 108.

Briefly turning to FIG. 6, a histogram of the determined second image intensity spectrum 130 for second zone 122 (e.g., outer zone) is depicted. As shown in FIG. 6, the image/pixel intensity spectrum 130 may include various image intensity data points organized in a histogram that may be collected when analyzing second zone 122 of single, combined image 124. Additionally, histogram of the second image intensity spectrum 130 may also include or depict the defined second image intensity threshold 132. As discussed herein, the defined second image intensity threshold 132 may be defined based on the determined second image intensity spectrum 130. For example, second image intensity threshold 132 may be defined, calculated, and/or determined based on a predetermined percentage of data or value points of the second image intensity spectrum 130 that are equal to or closest in value to a “0” (zero) image intensity. As shown in FIG. 6, second image intensity threshold may be defined or calculated as approximately “−75” image intensity as a result of only 5% (e.g., predetermined percentage) of the data or value points included in second image intensity spectrum 130 are equal to or above “−75”.

Returning to FIG. 5A-5C, once the respective image intensity spectrums and image intensity thresholds are determined/defined, single, combined image 124 may further undergo further processing during rewindowing. For example, single, combined image 124 may undergo a histogram matching or histogram equalization process to uniformly distribute the image intensity for each pixel across single, combined image 124. The histogram matching may use predetermined histograms for matching the histogram for each of the first image intensity spectrum and the second image intensity spectrum respectively. That is, the histogram matching process may match the first image intensity spectrum of the first zone with a first predetermined histogram, and may match the second image intensity spectrum of the second zone with a second predetermined histogram. In another non-limiting example, the histogram matching process performed during rewindowing may simply shift the image intensity values for each of the first image intensity spectrum and the second image intensity spectrum by a predetermined amount. In this non-limiting example, the image intensity data or values included in each of the first image intensity spectrum and the second image intensity spectrum may be shifted the same amount, or may be shifted distinct amounts. As result of performing the histogram matching process during rewindowing, an intermediate combined image 126 may be formed or generated, as shown in FIG. 5B. By comparison, the intermediate combined image 126 may be darker than single, combined image 124.

Additionally, rewindowing single, combined image 124 may also include further adjusting or manipulating single, combined image 124/intermediate combined image 126. For example, intermediate combined image 126 may further be adjusted and/or manipulated based on the defined first image intensity threshold and the second image intensity threshold. As such, an even dark/more contrast image, or final, single, combined image 128 (hereafter, “final”), combined image 128″) may be generated, formed, and/or created during the rewindowing process. Final, combined image 128, as shown in FIG. 5C, may undergo additional processes discussed herein for detecting lesion(s) in the imaged prostate. As similarly discussed herein, the processes of rewindowing single, combined image 124 to form intermediate, combined image 126 and final, combined image 128 as shown in FIGS. 5B and 5C, respectively, may be performed by computing device 106 and/or neural network 108 of system 100.

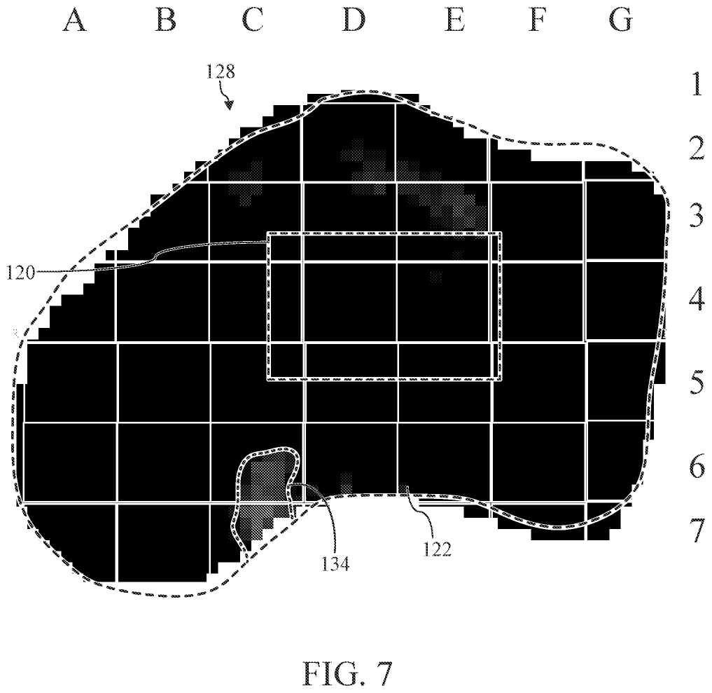

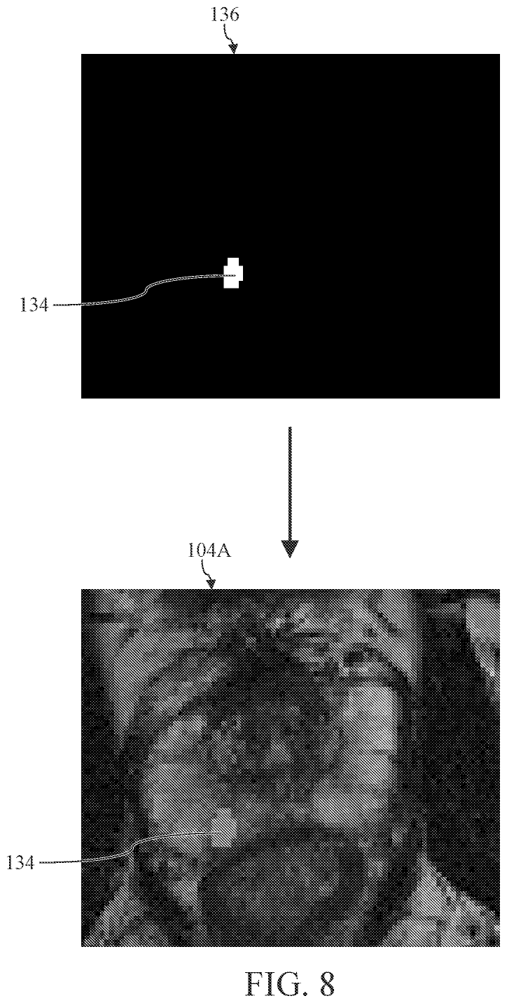

FIG. 7 shows a denoised final, combined image 128 similar to that shown in FIG. 5C. the process of denoising final, combined image 128 may improve image quality and in turn may increase the accuracy in detecting lesion(s) within the imaged prostate. In a non-limiting example, and as shown in FIG. 7, denoising of combined image 128 may include first dividing or sectioning the final, single, combined image 128 into a plurality of distinct regions. In the example, each of the plurality of distinct regions may associated or correspond with first zone 120 or second zone 122, or a combination thereof. As shown in FIG. 7, the plurality of regions may be formed as a grid pattern and may be labelled as an alphanumeric from A4-G7. Each of the plurality of regions A4-G7 may include a predetermined dimension or size. That is, and as shown in FIG. 7, each of the plurality of regions A4-G7 in final, combined image 128 may include a predetermined dimension or size, where full or complete regions (e.g., D4, E4, C5, F6, etc.) are evenly dimensioned. Other regions A4-G7 may not be complete and/or include a complete dimension of a region as a result of the shape/geometry of the imaged prostate. In a non-limiting example the predetermined dimension for each of the plurality of distinct (complete) regions may be 4×4×3.

Once divided, denoising singe, combined image 128 may include concatenating each of the plurality of regions A4-G7 to identify individual region image intensity values. More specifically, the image intensity of each of the plurality of images A4-G7 may be analyzed, calculated, and/or summed to define, determine, and/or identify image intensity values for each of the plurality of regions A4-G7. The summed image intensity values may represent the total image/pixel intensity for the entire area in each of the individual plurality of regions A4-G7. The identified image intensity values for each of the plurality of regions A4-G7 of single, combined image 128 may then be compared to the first image intensity threshold or second image intensity threshold based upon the region's association with the first zone 120 or second zone 122 of the imaged prostate. For example, region D4 of the plurality of regions A4-G7 may be compared to the first image intensity threshold, while region B6 may be compared to the second image intensity threshold 132 (see, FIG. 6). In other non-limiting examples where a region may span across and/or be formed in both the first zone 120 and second zone 122 (e.g., region C4), the sum or identified image intensity value for that region may be compared to both the first image intensity threshold or second image intensity threshold, or alternatively may be compared to the lower of the two image intensity thresholds.

The comparison between image intensity values and the image intensity thresholds in the denoising process may identify regions that may be equal to or exceed the threshold(s). More specifically, denoising single, combined image 128 by comparing the region image intensity values for each of the plurality of regions A4-G7 to the first/second image intensity threshold(s) may identify at least one positive region of the plurality of regions A4-G7 that may include an identified/summed image intensity that is equal to or exceeds the threshold(s). As discussed herein, the identification of at least one positive region may aid in the identification of a portion of the prostate that may include a detected lesion or lesions. With respect to FIG. 7, region F5 may have a summed and/or identified image intensity value that is less than the second image intensity threshold 132 (see, FIG. 6) determined during the rewindowing process. However, the identified/summed image intensity value of region C6 of the plurality of distinct regions A4-G7 may be greater than the second image intensity threshold 132. As such, region C6 may go through additional analysis to identify/confirm if a lesion has been detected within the portion of the imaged prostate that corresponds to region C6. Additionally, in the non-limiting example shown in FIG. 7, it may be determined that identified/summed image intensity value of region C7 is also equal to or greater than the second image intensity threshold 132.

Similar to other processes discussed herein (e.g., the rewindowing process), denoising single, combined image 128 may be performed and/or achieved by computing device 106 and/or neural network 108. For example, computing device 106 and/or neural network 108 may utilize open source software like Python to perform the process of dividing final, single, combined image 128 in the plurality of distinct regions.

Where the summed/identified image intensity value for every region of the plurality of regions A4-G7 is not identified or determined to be greater than or equal to the image intensity threshold(s), denoising of single, combine image 128 may include additional steps and/or processes. For example, in response to determining no region image intensity value for each of the plurality of regions A4-G7 is equal to or greater than the first image intensity threshold and/or the second image intensity threshold, the image intensity threshold(s) may be adjusted. That is, if no region image intensity is equal to or greater than the image intensity threshold(s), computing device 106 and/or neural network 108 may subsequently adjust the first image intensity threshold for first zone 120 (e.g., inner zone) of the prostate based on the first image intensity spectrum, and/or second image intensity threshold for second zone 122 (e.g., outer zone) of the prostate based on the second image intensity spectrum. Alternatively, the first image intensity threshold and/or the second image intensity threshold may be adjusted where it is determined that more regions than is likely or is medically probable (e.g., over 50% of regions) are equal to or exceed the respective thresholds. Once adjusted, the region image intensity value for each of the plurality of regions A4-G7 may be (re)compared to the adjusted first image intensity threshold and/or the adjusted second image intensity threshold to identify at least one region that may be equal to or exceeds the adjusted image intensity threshold(s).

A portion of the imaged prostate may subsequently be identified as including a detected lesions based on the identified region of the plurality of regions that is equal to or greater than the image intensity thresholds. That is, the identified region of the plurality of regions that includes an image intensity value that is equal to or greater than the first/second image intensity threshold may in turn also indicated/confirm that a lesion has been detected within the imaged prostate, and may further identify, indicate, and/or determine a portion of the imaged prostate that includes the detected lesion(s). In non-limiting examples, and based on the anatomical data relating to the imaged prostate, combined image 128, and/or plurality of defined zones 120, 122, the identified portion of the prostate including the detected lesion(s) may correspond to one of first zone 120 or second zone 122, and/or an anatomic location of the detected lesion(s) within the prostate.

With continued reference to FIG. 7, and the examples discussed herein, it may be determined that regions C6 and C7 include image intensity values that are greater than the second image intensity threshold 132 (see, FIG. 6) for single, combined image 128. Knowing the location or regions that include image intensity values that are greater than the second image intensity threshold 132 may in turn identify the portion of the imaged prostate that includes a detected lesion 134, as well as the associated or corresponding zone including lesion 134 (e.g., second/outer zone 122), and/or anatomic location (e.g., inferior, medial-left) of the prostate based on the anatomical date relating to the prostate.

Additionally, in non-limiting examples, identifying the portion of the prostate including the detected lesion 134 may include additional steps and/or processes. That is, each of identified regions C6 and C7 including the detected lesion 134 may be analyzed and/or undergo additional review and/or processing in order to accurately map the detected lesion on MRI images that may be presented to a user (e.g., MRI technician, radiologist, physician) for ease of review. For example, each region C6 and C7 may undergo additional segmentation, pixel division/analysis processes, and/or denoising processes to identify the portions within each region C6, C7 that include the detected lesion 134. As such, all pixels of image 128 associated with detected lesion 134 and included within regions C6 and C7 may be identified. By performing these additional processes, the shape and/or size of detected lesion 134 may be more accurately determined, calculated, and/or represented. As discussed herein, defining/calculating the shape and/or size of detected lesion 134 using the processes discussed herein may further aid in detecting and/or accurately diagnosing the imaged prostate. Additionally, by segmenting regions C6 and C7, detected lesion 134 may be outlined in final, single, combined image 128, as shown in FIG. 8.

Similar to other processes discussed herein, detecting lesion 134 and identifying the portion of the imaged prostate that includes detected lesion 134 may be performed and/or achieved by computing device 106 and/or neural network 108.

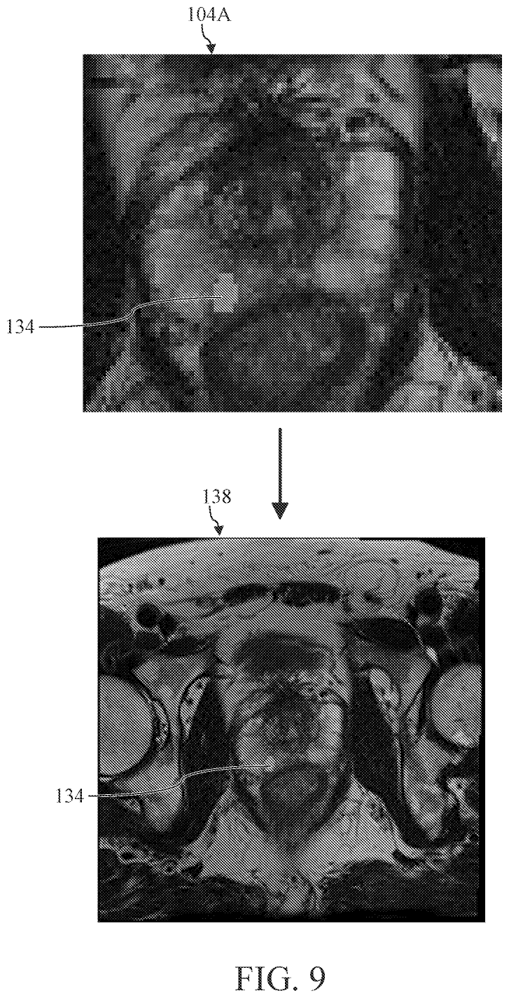

Once identified within single, combined image 128 as shown in FIG. 7, combined, image 128 including the detected/identified lesion 134 may be further manipulated. More specifically, combined, image 128 may adjusted such that all pixels of the image associated with or corresponding to detected lesion 134 may be adjusted to a first intensity (e.g., white), and all other pixels of image 128 may be adjusted to a second intensity (e.g., black). In a non-limiting example show FIG. 8, an adjusted image 136 of the prostate may include detected lesion 134 identified as/adjusted to white, while the remaining portions may be identified as/adjusted to black. Additionally as shown in FIG. 8, adjusted image 136 may subsequently be collapsed with, masked over, and/or included in an MRI image 104, for example MRI image 104A, to more clearly depict or display detected lesion 134 in an MRI image 104. Once included, masked, and/or collapsed with MRI image 104A, MRI image 104A including detected lesion 134 may be enhanced, collapsed, combined, and/or masked to form a final MRI image 138, as shown in FIG. 9.

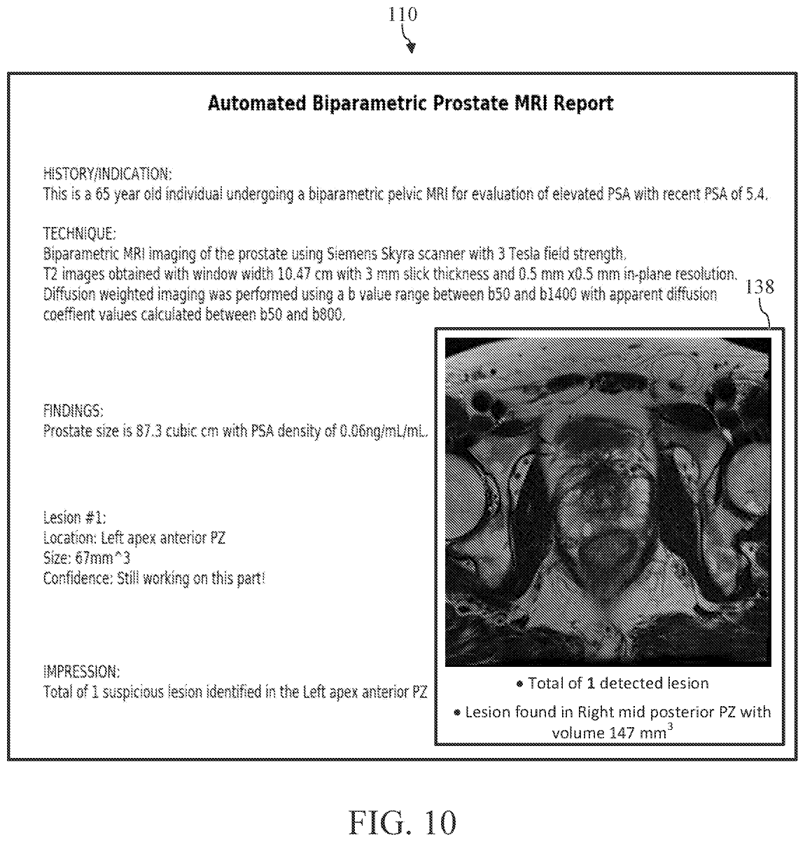

Subsequent to identifying the portion of the prostate that includes detected lesion 134 and/or the generation of final MRI image 138, processes for detecting lesions in imaged prostates may also include generating report 110. As previously discussed herein with respect to FIG. 1, and as shown in FIG. 10, report 110 may be generated based on the identifying of the portion of the prostate including detected lesion 134. Generated report 110 may include information, data, and/or images relating to the detection of lesion 134 in the imaged prostate. The information included in report 110 may aid in a user (e.g., MRI technician, radiologist, physician) to confirm the presence/diagnose lesion 134 within the prostate, and be provided a comprehensive set of information relating to the detection of the lesion 134. In non-limiting examples shown in FIG. 10, generated report 110 may include visual data relating to the identified portion of the prostate including the detected lesion 134 (e.g., final MRI image 138), as well as probabilities for respective risk categories associated with detect lesion 134, a determined/identified location of detected lesion 134, a size/shape of detected lesion 134, the number of lesions detected in the imaged prostate, patient information, and information relating to the MRI procedures/devices 102 used in order to obtain MRI images 104 of the patient's prostate. The location of the detected lesion may also include anatomic coordinates corresponding to the location of the detected lesion 134, where the set of anatomic coordinates are based on the anatomical data relating to the prostate and/or included within MRI images 104 originally created/generated by MRI device 102 of system 100 (see, FIG. 1). Additionally, generated report 110 may also include options for modifying report 110 itself and/or changing data/information provided therein. As discussed herein, report 110 may be generated as a physical print out, a graphical depiction provided on a display device of computing device 106 (e.g., screen monitor), or any other suitable visual representation providing information or data relating to the analysis of MRI images 104 and/or the detection of lesion(s) 134 in the image of patient's prostate. As similarly discussed herein with respect to other processes, generating report 110 may be performed and/or achieved by computing device 106 and/or neural network 108.

Although discussed herein with respect to FIG. 1-10 as being performed using a plurality of MRI images 104, it is understood that the process for detection lesions 134 using system 100 may be performed using only a single MRI image 104. All processes for detecting lesion 134 in the imaged prostate may be performed as similarly discussed herein, except the process of collapsing. That is, because only a single MRI image is being used to detect lesion 134, the collapsing process may not be performed, but rather the process may rewindow the single MRI image after first segmenting the single MRI image.

FIGS. 11-18 show magnetic resonance imaging (MRI) images 104 including distinct, defined zones. That is, FIGS. 11-18 show various non-limiting examples of distinct plurality of zones that may be formed and/or defined during the segmentation process of MRI image 104. The plurality of zones discussed herein may be distinct from, or alternatively may be formed concurrently with, inner zone and outer zone discussed herein with respect to FIG. 3B. It is understood that similarly numbered and/or named components may function in a substantially similar fashion. Redundant explanation of these components has been omitted for clarity.

Turning to FIG. 11, MRI image 104 may be segmented to define a plurality of zones 120, 122. In the non-limiting example, first zone 120 may include a superior zone of the imaged prostate, while the second zone 122 may include an inferior zone of the prostate. As shown, inferior zone corresponding to second zone 122 may be formed or defined substantial opposite and/or below the superior zone corresponding to first zone 120.

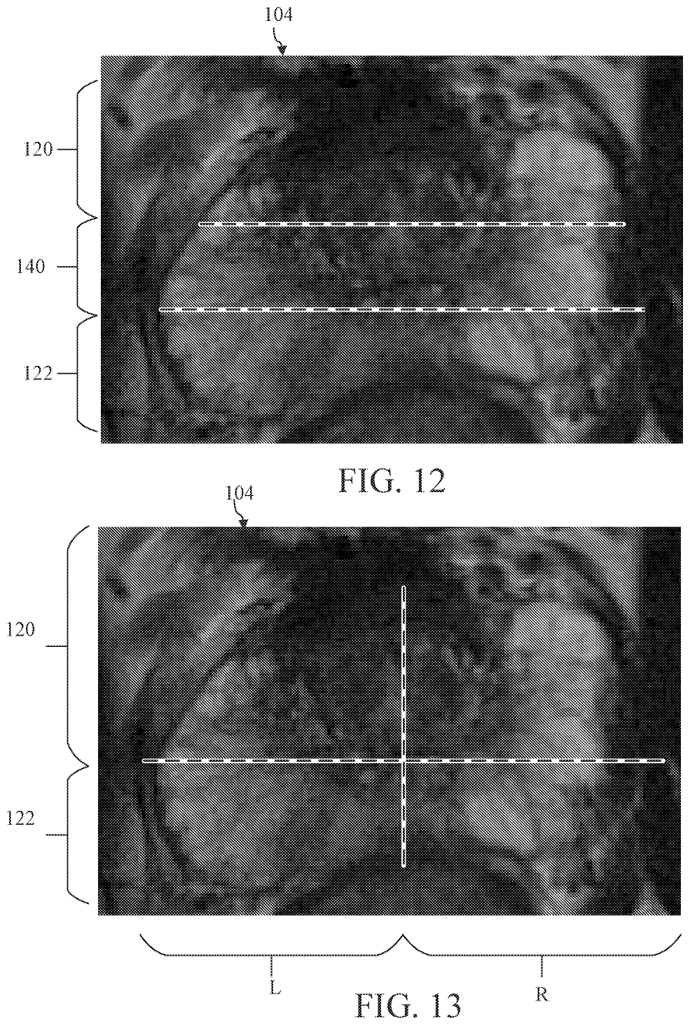

FIG. 12 shows another non-limiting example of MRI image 104 including a plurality of zones. Similar to FIG. 11, the example shown in FIG. 12 may include first zone 120 may include a superior zone of the imaged prostate, while the second zone 122 may include an inferior zone of the prostate. Additionally, the plurality of zones may include a third zone 140 distinct from first zone 120 and second zone 122, respectively. As shown in FIG. 12, third zone 140 may include a medial zone positioned substantially between superior zone corresponding to first zone 120 and inferior zone corresponding to second zone 122.

FIG. 13 shows another non-limiting example where MRI image 104 includes first zone 120 including a superior zone of the imaged prostate, and second zone 122 including inferior zone of the prostate. Furthermore, each of first zone 120 (e.g., superior zone) and second zone 122 (e.g., inferior zone) may further be divided and/or the plurality of zones may include a left zone (L) and a right zone (R). As such, and as shown in FIG. 13, first zone 120 may in fact include a left first zone 120L and a right first zone 120R, while second zone 122 may include a left second zone 122L and a right second zone 122R.

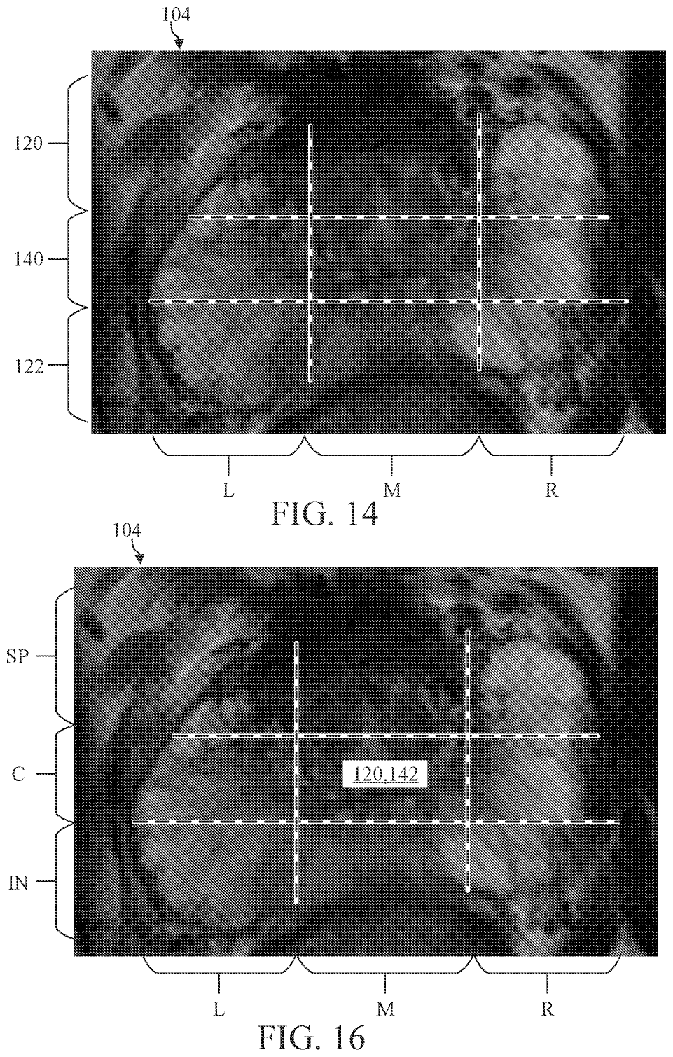

FIG. 14 is another non-limiting example that builds upon the previous non-limiting examples discussed herein with respect to FIGS. 11-13. That is, MRI image 104 shown in FIG. 14 may include first zone 120 formed as a superior zone of the imaged prostate, while the second zone 122 may include an inferior zone of the prostate. Additionally, MRI image 104 may include third zone 140 formed as a medial zone positioned substantially between superior zone corresponding to first zone 120 and inferior zone corresponding to second zone 122. Furthermore, each of first zone 120 (e.g., superior zone), second zone 122 (e.g., inferior zone), and third zone 140 (e.g., medial zone) may further be divided and/or the plurality of zones may include a left zone (L), a right zone (R), and a middle zone (M) formed between the left zone (L) and the right zone (R). As such, and as shown in FIG. 14, first zone 120 may in fact include a left first zone 120L, a right first zone 120R, and a middle first zone 120M. Additionally, second zone 122 may include a left second zone 122L, a right second zone 122R, and a middle second zone 122M. Finally, third zone 140 may include a left third zone 140L, a right third zone 140R, and a middle third zone 140M.

FIGS. 15A and 15B show further non-limiting examples of the plurality of zones 120, 122 formed in MRI image 104. In the example, defined first zone 120 shown in FIG. 15A may correspond to an anterior zone 142 of the imaged prostate. FIG. 15B may show a reverse image or side of the imaged prostate than that shown in FIG. 15A. In this example, defined second zone 122 shown in FIG. 15B may correspond to a posterior zone 144 of imaged prostate. Posterior zone 144 may be formed or defined opposite anterior zone 142/first side 120 (e.g., into the page in FIG. 15A). In this example, anterior zone 142/first zone 120 may define the front of the imaged prostate, which posterior zone 144/second zone 122 may define the back/rear of the imaged prostate. In another non-limiting example (not shown) MRI image 104 may define a third zone 140 corresponding to a medial zone formed between anterior zone 142 and positioner zone 144 (e.g., into the page in FIG. 15A or 15B).

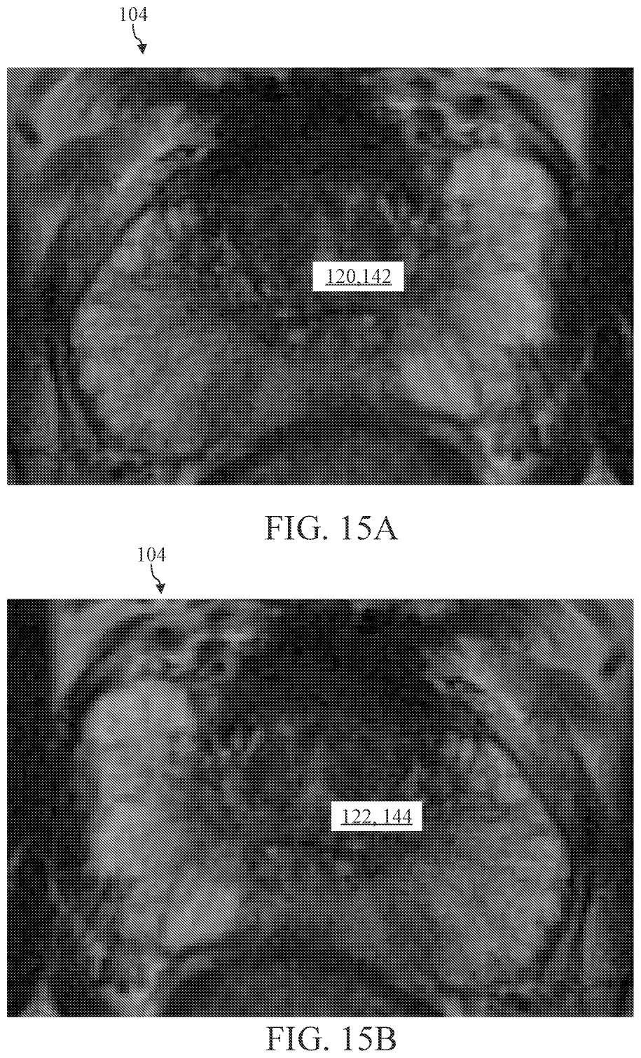

FIG. 16 shows yet another non-limiting example where MRI image 104 includes a plurality of defined zones. In this non-limiting example, anterior zone 142 formed as first zone 120 and the respective sub-portions/zones of anterior zone 142 are shown and discussed herein. However, it is understood that posterior zone 144 (see, FIG. 15B) and/or medial zone (not shown) may include similar sub-portions/zones. As shown in FIG. 16, anterior zone 142 formed as first zone 142 may include superior (SP) subzones, central subzones (C), inferior (IN) subzones, left (L) subzones, middle (M) subzones, and right (R) subzones. As such, anterior zone 142 may include a left-superior-anterior zone 142LSP (upper left), a left-central-anterior zone 142LC (central left), a left-inferior-anterior zone 142 LIN (lower left), a middle-superior-anterior zone 142MSP (upper middle), a middle-central-anterior zone 142MC (central middle), a middle-inferior-anterior zone 142 MIN (lower middle), a right-superior-anterior zone 142RSP (upper right), a right-central-anterior zone 142RC (central right), and a right-inferior-anterior zone 142 RIN (lower right).

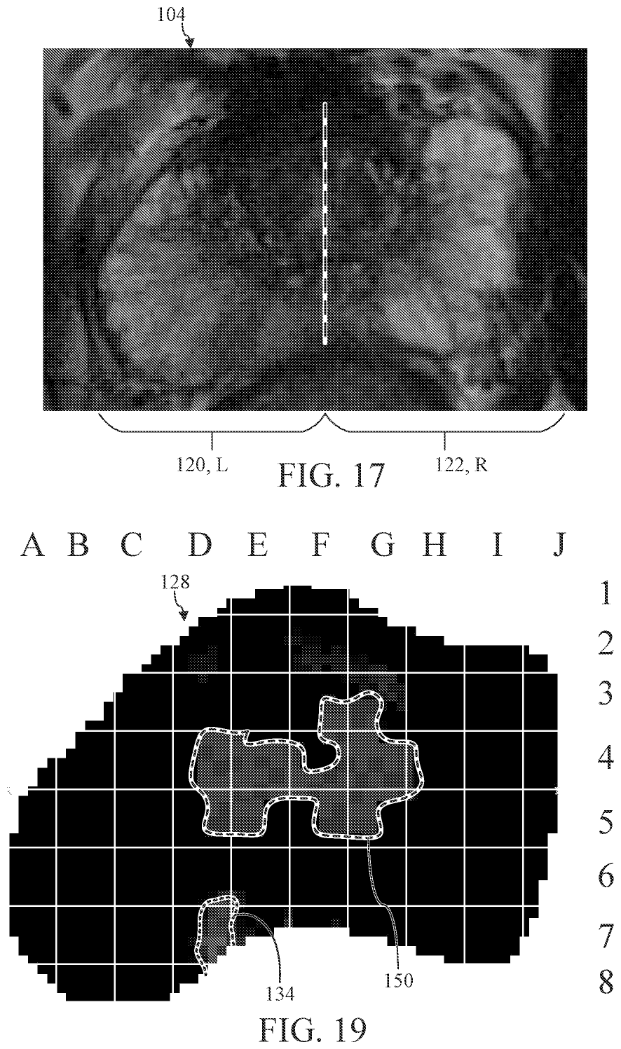

FIG. 17 show another non-limiting example of MRI image 104 including a plurality of defined zones 120, 122. In the non-limiting example, MRI image 104 may include first zone 120 including a left side (L) of the imaged prostate, and second zone 122 including right side (R) of imaged prostate. As shown, right side (R) corresponding to second zone 122 may be formed or defined substantial opposite and/or adjacent the left side (L) corresponding to first zone 120. In another non-limiting example (not shown), MRI image 104 may also include third zone 140 including a middle portion positioned between left side (L)/first zone 120 and right side (R)/second zone 122.

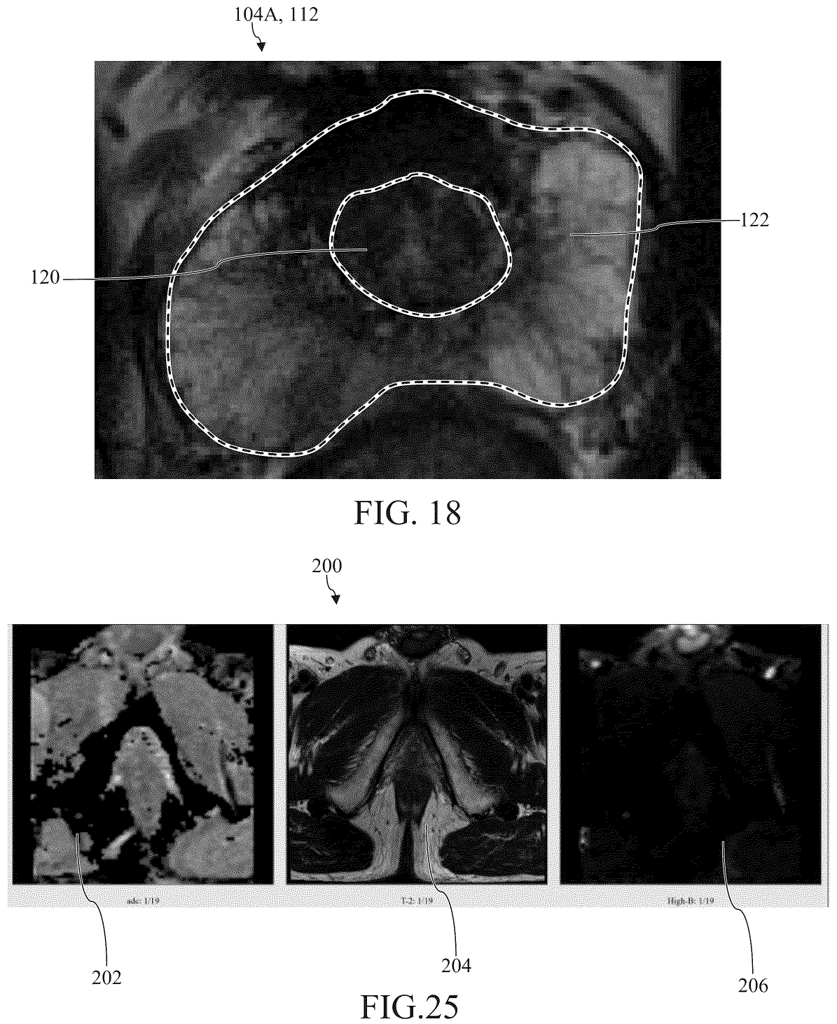

FIG. 18 shows a further non-limiting example of MRI image 104 including a plurality of defined zones 120, 122. In the non-limiting example, MRI image 104 may include first zone 120 corresponding to a transition zone of the prostate, and second zone 122 may correspond to a peripheral zone of the prostate. In this example, the peripheral zone forming second zone 122 may substantially surround the transition zone forming first zone 120.

It is understood that the plurality of zones identified and/or defined in the non-limiting examples shown in FIG. 11-18 are illustrative. As such, MRI image 104 may include any number and/or combination of zones defined during the segmentation process, as similarly discussed herein.

Although discussed herein as undergoing a segmentation process to define a plurality of distinct zones, it is understood that the segmentation process may be omitted when performing the processes for detecting lesions in an imaged patient's prostate. That is, the plurality of MRI images 104 may be collapsed into a single, combined image without performing the segmentation process thereon to define the plurality of distinct zones. As such, when performing the rewindowing process only a single image intensity and a single image intensity threshold may be determined and/or defined. However, because of known inherent distinctions in image intensities between different portions or zones (e.g., first zone v. second zone) of the imaged prostate, the process for identifying or detecting lesions may include additional steps to ensure accurate detection and/or identification of lesion(s) within the prostate. For example, and with reference to FIG. 19, after rewindowing and denoising the single, combined image 128, a validation process may be performed. More specifically, the identified portion of the prostate including detected lesion(s) 134, 150 may be validated to ensure the detections are accurate and/or correspond to a high likelihood of a lesion being present in the imaged prostate. Similar to the example discussed herein with respect to FIG. 7, detected lesion 134 included in regions D6, D7, D8, E6, and E7 may be confirmed as detected lesion based on a validation process. However, the alleged lesion 150 included in and/or spanning over regions D4, D5, E4, E5, F3, F4, F5, G3, G4, G5, H4, and H5 may undergo the validation process and subsequently determined to be a false positive and/or not a lesion. When validating alleged lesion 150, computing device 106/neural network 108 may analyze the anatomic location/regions of the prostate including the detected/alleged lesion 150, analyze a size of the detected-alleged lesion 150, and/or analyze the image intensity value for each of the regions D4, D5, E4, E5, F3, F4, F5, G3, G4, G5, H4, and H5 that are equal to or exceed the image intensity threshold and/or contain a portion of alleged lesion 150.

Once analyzed alleged lesion 150 may be identified as a false positive or false detection based on any of the analyzed data or information. In the non-limiting example shown in FIG. 19, alleged lesion 150 may be identified as a false positive or false detection based on the size and the location/anatomical data of the lesion itself. That is, alleged lesion 150 may be found in the first/inner zone, and may span or cover the majority (e.g., 80%) of the entire zone. Based on this obtained information, system 100, and more specifically computing device 106/neural network 108, performing the detection processes may be determined alleged lesion 150 is a false positive or false detection. To improve accuracy and/or prevent the detection of non-existent lesions, system 100 may adjust the image intensity thresholds and perform the denoising process again to eliminate the identification of alleged lesion 150. Additionally, or alternatively, and based on final MRI image 138, report 110, anatomical data associated with the prostate, and/or practical/medical knowledge of the prostate, a user (e.g., MRI technician, radiologist, physician) may choose to ignore or remove alleged lesion 150 from report 110 as easily being identified as a false positive or false detection. In the non-limiting example shown and discussed herein with respect to FIG. 19, the processes for identifying a false positive may be performed per patient, per MRI image 104, and/or per series of images 104A, 104B, 104C.

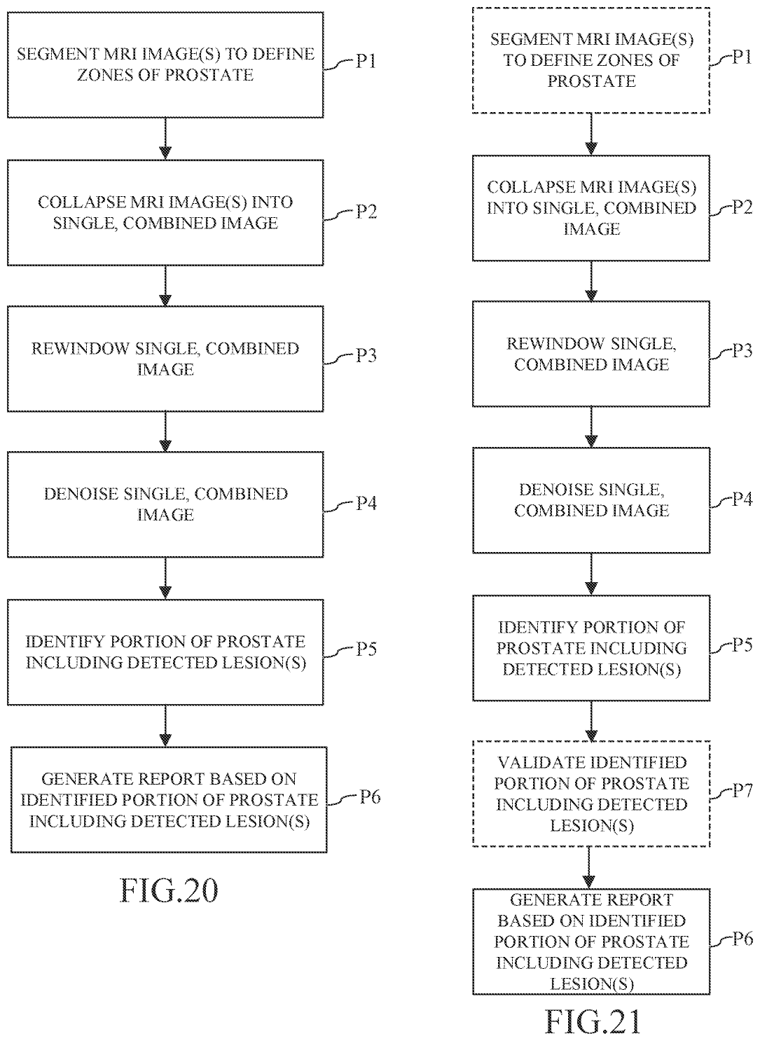

FIGS. 20-22 depicts non-limiting example processes for detecting lesions in a patient's prostate. Specifically, FIGS. 20-22 are flowcharts depicting various example processes for detecting lesions on a multiparametric prostate using magnetic resonance imaging (MRI). In some cases, a computing device(s) and/or system may be used to perform the processes for lesion detection, as discussed herein with respect to FIGS. 1 and/or 24.

Turning to FIG. 20, in process P1 a plurality of MRI images are segmented. More specifically, a plurality of obtained or generated MRI images of a patient's prostate are segmented to define a plurality of distinct zones or areas of the prostate. In a non-limiting example, the MRI images of the patient's prostate may be segmented two define a first zone of the prostate depicted in each of the plurality of MRI images and a second zone of the prostate depicted in each of the plurality of images. The second zone defined in process P1 may be distinct from the first zone. Additionally each of the first zone and the second zone may include anatomical data relating to the patient's prostate. In one example the first zone of the plurality of zones may include an inner zone of the prostate and the second zone of the plurality of zones may include an outer zone of the prostate. In another non-limiting example, the first zone may include a transition zone of the prostate and the second zone of the plurality of zones may include a peripheral zone of the prostate, where the peripheral zone substantially surrounds the transition zone.

In another example the first zone of the plurality of zones may include an interior zone of the prostate and the second zone of the plurality of zones may include a posterior zone of the prostate, where the posterior zone is formed substantially opposite the anterior zone. In other non-limiting examples the plurality of zones defined during these segmentation of the plurality of MRI images and process P1 may also include a third zone distinct from the first sound in the second zone. where the first zone in the second zone include an anterior zone an posterior zone, respectively, the third zone may include a medial zone positioned substantially between the anterior zone and the posterior zone. Moreover, the anterior zone of the prostate may include an anterior-superior portion, an anterior-inferior portion positioned opposite the anterior-superior portion, and an anterior-central portion positioned between the anterior-superior portion and the anterior-inferior portion. The posterior zone of the prostate includes a posterior-superior portion, a posterior-inferior portion positioned opposite the posterior-superior portion, and a posterior-central portion positioned between the posterior-superior portion and the posterior-inferior portion. Furthermore, the medial zone of the prostate may include a medial-superior portion, a medial-inferior portion positioned to opposite the medial-superior portion, and a medial-central portion positioned between the medial-superior portion and the medial-inferior portion.

In other non-limiting examples, the first zone of the plurality of zones may include a superior zone of the prostate and the second zone may include an inferior zone of the prostate. The inferior zone may be formed substantially opposite the superior zone. Additionally, the plurality of zones defined during the segmenting of the plurality of MRI images may further include defining a third zone distinct from the first zone in the second zone. The third zone may include a medial zone positioned substantially between the superior zone and the inferior zone. In this example, the superior zone of the prostate may also include a superior-anterior portion, a superior-posterior portion positioned opposite the superior-anterior portion, and a superior-central portion positioned between the superior-anterior portion and the superior-posterior portion. The inferior zone of the prostate may include an inferior-anterior portion, an inferior-posterior portion positioned opposite the inferior-anterior portion, and an inferior-central portion positioned between the inferior-anterior portion and the inferior-posterior portion. Additionally, the medial zone of the prostate may include a medial-anterior portion, a medial-posterior portion positioned opposite the medial anterior portion, and a medial-central portion positioned between the medial-anterior portion and the medial-posterior portion.

The plurality of MRI images segmented in process P1 may be in a DICOM format prior to segmentation. Additionally, each of the plurality of MRI images may include distinct image characteristics. For example, the plurality of MRI images may include a low intensity MRI-image ranging from approximately 0.7 T-1.2 T, a mid-intensity MRI-image ranging from approximately 1.3 T-1.9 T, and a high-intensity MRI-image ranging from 2.0 T-3.0 T. Although segmented to define the plurality of distinct zones is based on, at least in part, the anatomical data relating to the prostate, process P1 may also include adjusting at least one of the plurality of zones of the prostate based on additional user input.

In process P2 the plurality of MRI images may be collapsed. More specifically, the plurality of MRI images may be collapsed, combined, and/or masked to form a single, combined image. Collapsing the plurality of MRI images to form the single, combined image may further include aligning each of the plurality of MRI images based on the zones defined during the segmentation process of P1. For example where the zones include the first zone and the second zone, each of the low intensity MRI-image, the mid-intensity MRI-image, and the high-intensity MRI-image may be aligned based on the defined first zone and the defined second zone in each of the respective images. Furthermore, the collapsing of the plurality of MRI images may include normalizing each of the aligned plurality of MRI images, and inverting a high-b value for each of the aligned plurality of MRI images. Additionally, and subsequent two inverting the high-b value, the aligned, plurality of MRI images may be segmented, and finally the segmented and aligned plurality of MRI images may be inverted.

In process P3 the single combined image may be rewindowed. That is, process P3 may include rewindowing the single, combined image formed from the plurality of MRI images by determining a first image intensity spectrum for the first zone of the prostate depicted in the single, combined image, and determining a second image intensity spectrum for the second zone of the prostate depicted in a single combined image. Additionally, rewindowing of the single, combined image may include defining a first image intensity threshold for the first zone of the prostate based on the first image intensity spectrum, and defining a second image intensity threshold for the second zone of the prostate based on the second image intensity spectrum.

In process P4 the single combined image may be denoised. That is, after rewindowing the single, combined image, the rewindowed single combined image may be denoised by first dividing the single, combined image into a plurality of distinct regions. Each of the plurality of regions may be associated with one of the first zone or the second zone of the prostate. Once divided, denoising the single, combined image may also include concatenating each of the plurality of regions to identify individual region image intensity values. The region image intensity values for each of the plurality of regions may then be compared to the first image intensity threshold or the second image intensity threshold based upon the region's association with either the first zone or the second zone of the prostate. Finally denoising the single, combined image may include identifying at least one positive region of the plurality of regions where the region image intensity value for the at least one positive region is equal to or greater than the first image intensity threshold or the second image intensity threshold. In the non-limiting example, each of the plurality of distinct regions may include a predetermined dimension. For example, the predetermined dimension for each of the plurality of distinct regions formed during the denoising process of P4 may be 4×4×3.

In response to not identifying at least one positive region in process P4 denoising the single, combined image may include additional steps and/or processes. More specifically, in response to determining no region image intensity value for each of the plurality of regions is equal to or greater than the first image intensity threshold or the second image intensity threshold, denoising the single, combined image in process P4 may include adjusting the first image intensity threshold for the first zone of the prostate based on the first image intensity spectrum and/or adjusting the second image intensity threshold for the second zone of the prostate based on the second image intensity spectrum. Once adjusted, the denoising process P4 may also include comparing the region image intensity value for each of the plurality of regions to the adjusted first image intensity threshold or the adjusted second image intensity threshold. The adjusting and comparison steps or processes may be performed until at least one positive region of the plurality of regions is identified.

In process P5 a portion of the prostate including a detected lesion(s) may be identified. More specifically, a portion of the prostate including a detected lesion(s) may be identified based on the identified at least one positive region of the plurality of regions (e.g., process P4). The identified portion of the prostate including the detected lesion(s) may correspond to and/or may identify the first zone or the second zone of the plurality of zones which may include the detected lesion(s), and/or an anatomic location of the prostate that may include the detected lesion(s). The anatomic location may be based on, for example, the anatomical data relating to the prostate.

In process P6 a report may be generated. More specifically, a report based on the identified portion of the prostate including the detected lesion(s) may be generated, created, displayed, and/or (physically) provided to a physician/MRI technician for review. The generated report may include at least one of visual data relating to the identified portion of the prostate including the detected lesion(s), probabilities for respective risk categories associated with the detected lesion(s), future procedures/actions to be taken on the patient's prostate, or options for modifications for the report. Additionally, or alternatively, the generated report may further include a set of anatomic coordinates corresponding to the location of a detected lesion on the prostate, where the set of anatomic coordinates are based on the anatomical data relating to the prostate.

FIG. 21 depicts another non-limiting example of processes for detecting lesions in a patient's prostate. It is understood that similarly numbered and/or named components/processes may function in a substantially similar fashion. Redundant explanation of these components has been omitted for clarity.

The processes shown in FIG. 21 may be substantially similar to those shown and discussed herein with respect to FIG. 20. That is, the processes shown in FIG. 21 may include processes P1-P6 substantially similar to processes P1-P6 of FIG. 20. However, and comparing the two processes, the flowchart shown in FIG. 21 depicts process P1 in phantom as optional. That is, the process of segmenting the plurality of MRI image(s) to define a plurality of distinct zones or areas of the prostate may be optional, and/or may not be performed prior to performing process P2. The processes for detecting lesions in a patient's prostate shown in FIG. 21, and discussed herein, may be described without performing process P1.

If process P1 is not performed, the process of detecting lesion(s) in a patient's prostate may begin at process P2. In process P2 the plurality of MRI images may be collapsed. More specifically, the plurality of MRI images may be collapsed, combined, and/or masked to form a single, combined image. Collapsing the plurality of MRI images to form the single, combined image may further include aligning each of the plurality of MRI images based know/defined anatomical data of the prostate depicted in the plurality of images. For example, each of the low intensity MRI-image, the mid-intensity MRI-image, and the high-intensity MRI-image may be aligned based on the anatomical data for reach image. Furthermore, the collapsing of the plurality of MRI images may include normalizing each of the aligned plurality of MRI images, and inverting a high-b value for each of the aligned plurality of MRI images. Additionally, and subsequent two inverting the high-b value, the aligned, plurality of MRI images may be segmented, and finally the segmented and aligned plurality of MRI images may be inverted.

In process P3 the single combined image may be rewindowed. That is, process P3 may include rewindowing the single, combined image formed from the plurality of MRI images by determining an image intensity spectrum for the prostate depicted in the single, combined image. Additionally, rewindowing of the single, combined image may include defining an image intensity threshold based on the image intensity spectrum. Unlike the processes discussed herein with respect to FIG. 20, the rewindowing process P3 in FIG. 21, where the images are not segmenting, may result in the determining of a single image intensity spectrum for the single, combined image, and a single defined image intensity threshold.

In process P4 the single combined image may be denoised. That is, after rewindowing the single, combined image, the rewindowed single, combined image may be denoised by first dividing the single, combined image into a plurality of distinct regions. That is, the entirety of the single, combined image may be divided into a plurality of regions. Once divided, denoising the single, combined image may also include concatenating each of the plurality of regions to identify individual region image intensity values. The region image intensity values for each of the plurality of regions may then be compared to the image intensity threshold. Finally, denoising the single, combined image may include identifying at least one positive region of the plurality of regions where the region image intensity value for the at least one positive region is equal to or greater than the image intensity threshold. In the non-limiting example, each of the plurality of distinct regions may include a predetermined dimension. For example, the predetermined dimension for each of the plurality of distinct regions formed during the denoising process of P4 may be 4×4×3.

In process P5 a portion of the prostate including a detected lesion(s) may be identified. More specifically, a portion of the prostate including a detected lesion(s) may be identified based on the identified at least one positive region of the plurality of regions (e.g., process P4). The identified portion of the prostate including the detected lesion(s) may correspond to and/or may identify the at least one zone of the plurality of zones which may include the detected lesion(s), and/or an anatomic location of the prostate that may include the detected lesion(s). The anatomic location may be based on, for example, the anatomical data relating to the prostate.

Process P7 is shown in phantom as optional. In one non-limiting example, process P7 may not be performed and process P6 may subsequently follow process P5. In process P6 a report may be generated. More specifically, a report based on the identified portion of the prostate including the detected lesion(s) may be generated, created, displayed, and/or (physically) provided to a physician/MRI technician for review. The generated report may include at least one of visual data relating to the identified portion of the prostate including the detected lesion(s), probabilities for respective risk categories associated with the detected lesion(s), future procedures/actions to be taken on the patient's prostate, or options for modifications for the report. Additionally, or alternatively, the generated report may further include a set of anatomic coordinates corresponding to the location of a detected lesion on the prostate, where the set of anatomic coordinates are based on the anatomical data relating to the prostate.

Alternatively, process P7 may be performed prior to performing process P6. In process P7, the identified portion of the prostate including the detected lesion(s) may be validated. That is, process P7 may perform a validation process regarding the detection/identification of a lesion(s) within the prostate. In a non-limiting example, validating the identified portion of the prostate including the detected lesion(s) may include analyzing the anatomic location (e.g., first zone, second zone, third zone, etc.) of the prostate including the detecting lesion(s), analyzing a size of the detected lesion, and/or analyzing the image intensity value for the at least one positive region that is equal to or greater than the image intensity threshold. Once analyzed, the detected lesion(s) may be subsequently identified as a false positive in response to analyzing the anatomic location, the size, and/or the image intensity. For example, when it is determined that the detected lesion is anatomically located in an area associated with the first zone, the size is approximately the size of 50% of the first zone, and/or the image intensity value is greater than the image intensity threshold by 30%, than the identified portion of the prostate including the detected lesion may be identified as a false positive. Subsequent to identifying a detected lesion as a false positive, processes P1, P2, P3, P4, and/or P5 may be adjusted and/or performed again in order to improve the detection of a lesion(s) within the patient's prostate, as discussed herein.

In response to not identifying at least one positive region in process P4, and/or validating the identified portion of the prostate results in the identification of the lesion(s) as a false positive in process P7, additionally processes may be performed. More specifically, in response to determining no region image intensity value for each of the plurality of regions is equal to or greater than the image intensity threshold, and/or identifying the detected lesion(s) as a false positive, process P4 may (or may be performed again to) adjust the image intensity threshold based on the image intensity spectrum. Once adjusted, the denoising process P4 may also include comparing the region image intensity value for each of the plurality of regions to the adjusted image intensity threshold. The adjusting and comparison steps or processes may be performed until at least one positive region of the plurality of regions is identified. Subsequently, the validation processes in process P7 may be subsequently performed using the adjusted image intensity threshold.

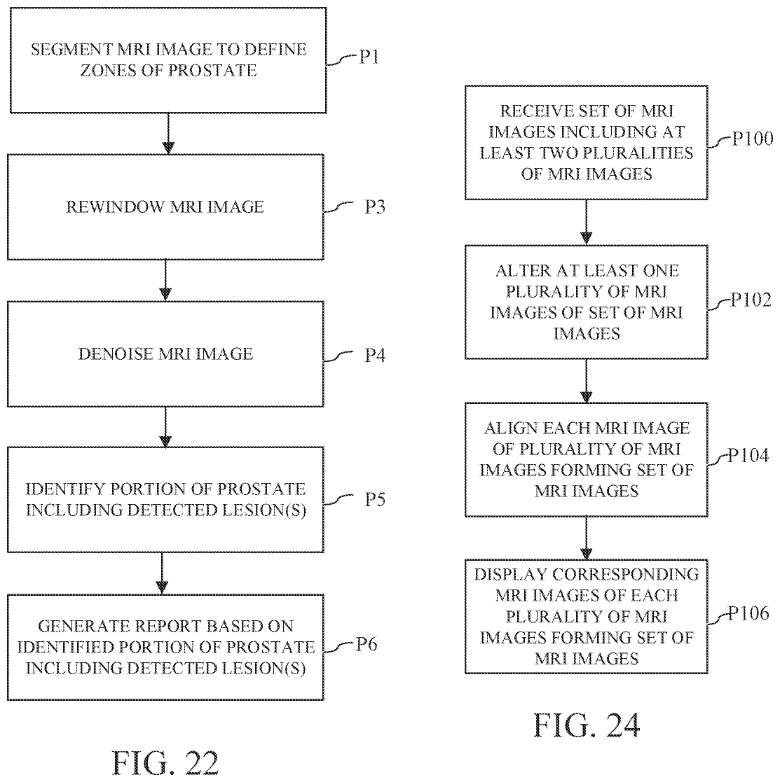

The processes shown in FIG. 22 may be substantially similar to those shown and discussed hercin with respect to FIGS. 20 and/or 21. That is, the processes shown in FIG. 22 may include processes P1 and P3-P6 substantially similar to processes P1 and P3-P6 of FIG. 20. However, and as discussed herein, the flowchart shown in FIG. 22 omits process P2. That is, the process of collapsing the plurality of MRI image(s) may not be performed in the process shown in FIG. 22. As a result of FIG. 22, depicts a process for detecting lesion(s) based on a single MRI image.

In process P1 the single MRI image is segmented. More specifically, an obtained or generated MRI image of a patient's prostate is segmented to define a plurality of distinct zones or areas of the prostate. In a non-limiting example, the MRI images of the patient's prostate may be segmented two define a first zone of the prostate depicted in each of the plurality of MRI images and a second zone of the prostate depicted in each of the plurality of images. The second zone defined in process P1 may be distinct from the first zone. Additionally, each of the first zone and the second zone may include anatomical data relating to the patient's prostate. Non-limiting examples of the first zone and second zone defined in the segmentation process are similarly discussed herein with respect to process P1 of FIG. 20. The MRI image segmented in process P1 may be in a DICOM format prior to segmentation. The MRI image may include a low intensity MRI-image ranging from approximately 0.7 T-1.2 T, a mid-intensity MRI-image ranging from approximately 1.3 T-1.9 T, or a high-intensity MRI-image ranging from 2.0 T-3.0 T. Although segmented to define the plurality of distinct zones is based on, at least in part, the anatomical data relating to the prostate, process P1 may also include adjusting at least one of the plurality of zones of the prostate based on additional user input.

In process P3 the single MRI image may be rewindowed. That is, process P3 may include rewindowing the single MRI image by determining a first image intensity spectrum for the first zone of the prostate depicted in the MRI image, and determining a second image intensity spectrum for the second zone of the prostate depicted in the single MRI image. Additionally, rewindowing of the MRI image may include defining a first image intensity threshold for the first zone of the prostate based on the first image intensity spectrum, and defining a second image intensity threshold for the second zone of the prostate based on the second image intensity spectrum.

In process P4 the single MRI image may be denoised. That is, after rewindowing the MRI image, the rewindowed single MRI image may be denoised by first dividing the MRI image into a plurality of distinct regions. Each of the plurality of regions may be associated with one of the first zone or the second zone of the prostate. Once divided, denoising the single MRI image may also include concatenating each of the plurality of regions to identify individual region image intensity values. The region image intensity values for each of the plurality of regions may then be compared to the first image intensity threshold or the second image intensity threshold based upon the region's association with either the first zone or the second zone of the prostate. Finally, denoising the MRI image may include identifying at least one positive region of the plurality of regions where the region image intensity value for the at least one positive region is equal to or greater than the first image intensity threshold or the second image intensity threshold. In the non-limiting example, each of the plurality of distinct regions may include a predetermined dimension. For example, the predetermined dimension for each of the plurality of distinct regions formed during the denoising process of P4 may be 4×4×3.

In response to not identifying at least one positive region in process P4 denoising the single MRI image may include additional steps and/or processes. More specifically, in response to determining no region image intensity value for each of the plurality of regions is equal to or greater than the first image intensity threshold or the second image intensity threshold, denoising the MRI image in process P4 may include adjusting the first image intensity threshold for the first zone of the prostate based on the first image intensity spectrum and/or adjusting the second image intensity threshold for the second zone of the prostate based on the second image intensity spectrum. Once adjusted, the denoising process P4 may also include comparing the region image intensity value for each of the plurality of regions to the adjusted first image intensity threshold or the adjusted second image intensity threshold. The adjusting and comparison steps or processes may be performed until at least one positive region of the plurality of regions is identified.

In process P5 a portion of the prostate including a detected lesion(s) may be identified. More specifically, a portion of the prostate including a detected lesion(s) may be identified based on the identified at least one positive region of the plurality of regions (e.g., process P4). The identified portion of the prostate including the detected lesion(s) may correspond to and/or may identify the first zone or the second zone of the plurality of zones which may include the detected lesion(s), and/or an anatomic location of the prostate that may include the detected lesion(s). The anatomic location may be based on, for example, the anatomical data relating to the prostate.

In process P6 a report may be generated. More specifically, a report based on the identified portion of the prostate including the detected lesion(s) may be generated, created, displayed, and/or (physically) provided to a physician/MRI technician for review. The generated report may include at least one of visual data relating to the identified portion of the prostate including the detected lesion(s), probabilities for respective risk categories associated with the detected lesion(s), future procedures/actions to be taken on the patient's prostate, or options for modifications for the report. Additionally, or alternatively, the generated report may further include a set of anatomic coordinates corresponding to the location of a detected lesion on the prostate, where the set of anatomic coordinates are based on the anatomical data relating to the prostate.

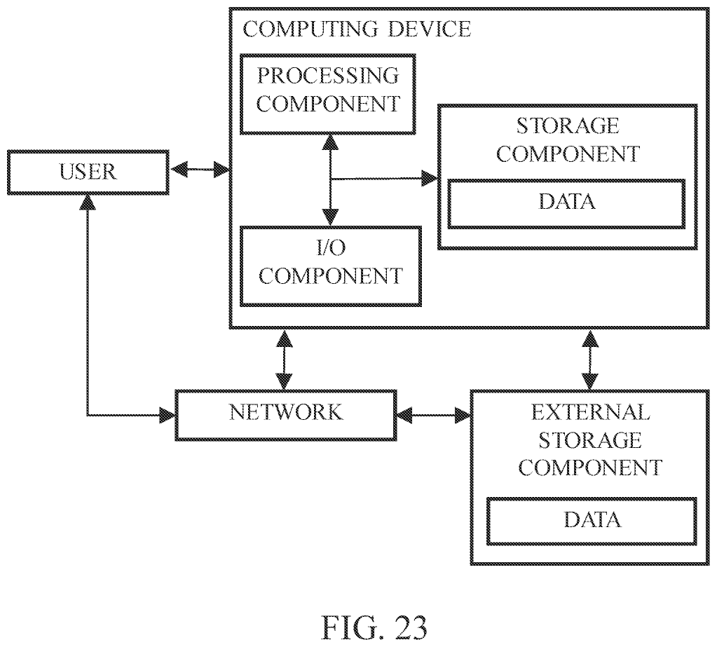

FIG. 23 depicts a schematic view of a computing environment or system (hereafter, “computing system”), and the various components included within computing system. In the non-limiting example shown in FIG. 23, computing system may include at least one computing device that may be configured to detect lesions on a prostate of a patient by performing the processes P1-P7 discussed herein with respect to FIGS. 20-22. It is understood that similarly numbered and/or named components may function in a substantially similar fashion. Redundant explanation of these components has been omitted for clarity.

It is understood that computing device(s) may be implemented as a computer program product stored on a computer readable storage medium. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Python, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and/or computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Computing system shown in FIG. 23 may include any type of computing device(s) and for example includes at least one processor or processing component(s), storage component, input/output (I/O) component(s) (including a keyboard, touchscreen, or monitor display), and a communications pathway. In general, processing component(s) execute program code which is at least partially fixed or stored in storage component. While executing program code, processing component(s) can process data, which can result in reading and/or writing transformed data from/to storage component and/or I/O component(s) for further processing. The pathway provides a communications link between each of the components in computing device(s). I/O component can comprise one or more human I/O devices, which enables user to interact with computing device(s) to analyze received MRI image(s) and detect lesions in a patient's prostate, as discussed herein. Computing device(s) may also be implemented in a distributed manner such that different components reside in different physical locations.

Storage component may also include modules, data and/or electronic information relating to various other aspects of computing system. Specifically, operational modules, electronic information, and/or data relating to media post MRI data, image segmentation data, image collapsing data, image rewindowing data, image denoising data, and report generation data. The operational modules, information, and/or data may include the required information and/or may allow computing system, and specifically computing device, to perform the processes discussed herein for detecting lesions in a patient's prostate.

Computing system, and specifically computing device of computing system, may also be in communication with external storage component. External storage component may be configured to store various modules, data and/or electronic information relating to various other aspects of computing system, similar to storage component of computing device(s). Additionally, external storage component may be configured to share (e.g., send and receive) data and/or electronic information with computing device(s) of computing system. In the non-limiting example shown in FIG. 23, external storage component may include any or all of the operational modules and/or data shown to be stored on storage component. Additionally, external storage component may also include a secondary database that user may interact with, provide information/data to, and/or may include information/data relating to poster. In a non-limiting example, external storage component may be a cloud-based storage component or system. In other non-limiting examples, external storage component may also include and/or be in communication with a neural network to aid in computation and/or data processing as discussed herein.

In a non-limiting example shown in FIG. 23, computing device(s) may be in communication with and/or may be configured to share (e.g., send and receive) data and/or electronic information over a network. Network may represent a closed network, such as a local area network (LAN) or may include the internet. Network may also include secondary database including similar data as storage component, and/or may include or be in communication with a neural network to aid in computation and/or data processing as discussed herein.

FIG. 24 shows a flowchart depicting non-limiting example processes of adjusting or modifying a set of radiology images. As discussed herein, the processes discussed herein may utilize similar alignment processes as discussed herein with respect to FIGS. 1-23 to adjust, optimize, and/or modify a set of radiology/MRI images, where each plurality of radiology/MRI images forming the set are captured with distinct image characteristics, parameters, and/or properties.

In process P100 a set of MRI images may be received. More specifically, a set of MRI images may be obtained, received, and/or provided subsequent to being captured. In a non-limiting example, the set of MRI images may capture images of a patient's prostate using an MRI machine or system, as discussed herein. The set of MRI images may include at least a first plurality of MRI images including first image characteristics, and a second plurality of MRI images having second image characteristics. The second image characteristics of the second plurality of MRI images of the set may be distinct from the first image characteristics of the first plurality of MRI images. Additionally, and as a result of the distinct image characteristics, the second plurality of MRI images may include or have a distinct number of MRI images compared to the first plurality of MRI images. First image characteristics and second image characteristics may include voxel parameters for each MRI image of the first plurality of MRI images and the second plurality of MRI images. For example, voxel parameters for each MRI image of the first plurality of MRI images may include 0.3 millimeters (mm)×0.3 mm×3 mm, resulting in a total of 30 images (or slices) being included in the first plurality of MRI images. Additionally in the example, voxel parameters for each MRI image of the second plurality of MRI images may include 0.5 millimeters (mm)×0.5 mm×2 mm, resulting in a total of 25 images (or slices) being included in the second plurality of MRI images.

In process P102, at least a portion of the set of MRI images may be altered. More specifically, the first plurality of MRI images and/or the second plurality of MRI images of the set of MRI images may be altered based on the first image characteristics and/or the second image characteristics. Subsequent to the altering of the first and/or second plurality of MRI images, the number of images in the first plurality of MRI images may be equal to the number of images in the second plurality of MRI images. Additionally, each of the equal number of MRI images in the first and second plurality of MRI images may correspond with one another. That is, a first MRI image of the first plurality of MRI images may correspond to, depict similar features, and/or visually display a similar portion of the patient's prostate as a first MRI image of the second plurality of MRI images. Furthermore, subsequent MRI images of the first plurality of MRI images may correspond to subsequent MRI images of the second plurality of MRI images as well.

Altering the first plurality of MRI images and/or the second plurality of MRI images of the set of MRI images in process P102 may include and/or may be achieved by adjusting the first image characteristics of the first plurality of MRI images and/or the second image characteristics of the second plurality of MRI images to be equal to one another. For example, voxel parameters (e.g., image characteristics) for each MRI image of the first plurality of MRI images may be adjusted from 0.3 millimeters (mm)×0.3 mm×3 mm to 0.1 millimeters (mm)×0.1 mm×1 mm. Additionally, voxel parameters for each MRI image of the second plurality of MRI images may be adjusted from 0.5 millimeters (mm)×0.5 mm×2 mm to 0.1 millimeters (mm)×0.1 mm×1 mm as well. Once adjusted, the first plurality of MRI images and/or the second plurality of MRI images may undergo an interpolation process based on the adjusted first image characteristics and/or the adjusted second image characteristics. The interpolation process may include altering, adjusting, and/or transforming the image content and/or image properties of at least one MRI image of the first plurality of MRI images and/or the second plurality of MRI images. Additionally, or alternatively, the interpolation process may include creating at least one new MRI image of the first plurality of MRI images and/or the second plurality of MRI images, and/or may include remove at least one existing MRI image of the first plurality of MRI images and/or the second plurality of MRI images. Interpolation of the first and/or second plurality of MRI images may be achieved, for example, using any suitable program product, software, artificial intelligence (AI) and/or neural network configured to manipulate the MRI images as discussed herein.

In process P104 each MRI image of the first plurality of MRI images may be aligned with the corresponding MRI image of the second plurality of MRI images. That is, and subsequent to the altering of the first and/or second plurality of MRI images, each MRI image of the first plurality of MRI images may be aligned, repositioned, and/or adjusted to match the image content and/or image properties of the corresponding MRI of the second plurality of MRI images. In non-limiting examples, MRI images of the first and second plurality of MRI images may be aligned using similar processes as discussed herein with respect to FIGS. 1-23.

In process P106 MRI images of the set of MRI images for the patient may be displayed. More specifically, and subsequent to the altering of at least one MRI image of the set of MRI images in process P102, the first MRI image of the first plurality of MRI images and the corresponding first MRI image of the second plurality of MRI images may be displayed, presented, and/or visually depicted adjacent to one another. Additionally, each subsequent MRI image of the first plurality of MRI images and the corresponding subsequent MRI images of the second plurality of MRI images may be displayed, presented, and/or visually depicted adjacent one another as well. In a non-limiting example, when a user (e.g., diagnosing physician or MRI technician) viewing the MRI images changes from the first MRI image of the first plurality of MRI images to a distinct MRI image, the corresponding MRI image of the second plurality of MRI images may also (automatically) change as well. As such, the user may view corresponding MRI images having different image characteristics or properties, but each corresponding MRI image may display identical portions and/or features of the patient's imaged body part (e.g., prostate).

Although two distinct pluralities of MRI images are discussed herein, it is understood that the set of MRI images may include more pluralities of MRI images. For example, set of MRI images may also include a third plurality of MRI images having a distinct number of MRI images than the first plurality of images and/or the second plurality of images. The third plurality of MRI images may include third image characteristics that may be distinct from the first image characteristics of the first plurality of MRI images and/or the second image characteristics of the second plurality of MRI images. As similarly discussed herein, the third plurality of MRI images may undergo the receiving steps of process P100, altering step of process P102, alignment step of process P104, and display step of process P106. Redundant explanation of these processes has been omitted for clarity/brevity.

FIG. 25 shows a non-limiting example of a user's graphical interface 200 showing a plurality of MRI images forming a set of MRI images. More specifically, graphical interface 200 depicts a first MRI image 202 of a first plurality of MRI images, a corresponding first MRI image 204 of a second plurality of MRI images, and a corresponding first MRI image 206 of a third plurality of MRI images—each of the first, second, and third plurality of MRI images forming the set of MRI images for a patient. In the non-limiting example, first MRI images 202, 204, 206 may have already undergone the modification processes (e.g., P1-P4) as discussed herein with respect to FIG. 24. That is, at least one MRI image of the first plurality of MRI images, the second plurality of MRI images, and/or the third plurality of MRI images may be altered to improve the viewability and/or aid a user (e.g., physician, MRI technician) in diagnosing any abnormalities and/or diseases as depicted in the MRI images.

In a non-limiting example, first plurality of MRI images and second plurality of MRI images may be captured using image characteristics (e.g., voxel parameters) of 0.3 millimeters (mm)×0.3 mm×3 mm, resulting in a total of 30 images (or slices). Additionally in the example, voxel parameters for each MRI image of the second plurality of MRI images may include 0.5 millimeters (mm)×0.5 mm×5 mm, resulting in a total of 25 images. After undergoing the image modification process discussed herein (see, FIG. 24), at least one MRI image of the first, second, and/or third plurality of MRI images may be altered (e.g., modified, manipulated, remove, and/or newly created images) such that each of the first, second, and third plurality of images may include 19 MRI images each. Each corresponding image of the 19 MRI images included in the first, second, and third plurality of MRI images may include, display, and/or depict identical or the same features of the patient's images body-part (e.g., prostate), such that a user viewing the images may view the same features under different imaging characteristics to improve diagnosis methods.

The flowchart and block diagrams in the Figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As discussed herein, various systems and components are described as “obtaining” data. It is understood that the corresponding data can be obtained using any solution. For example, the corresponding system/component can generate and/or be used to generate the data, retrieve the data from one or more data stores (e.g., a database), receive the data from another system/component, and/or the like. When the data is not generated by the particular system/component, it is understood that another system/component can be implemented apart from the system/component shown, which generates the data and provides it to the system/component and/or stores the data for access by the system/component.

The foregoing drawings show some of the processing associated according to several embodiments of this disclosure. In this regard, each drawing or block within a flow diagram of the drawings represents a process associated with embodiments of the method described. It should also be noted that in some alternative implementations, the acts noted in the drawings or blocks may occur out of the order noted in the figure or, for example, may in fact be executed substantially concurrently or in the reverse order, depending upon the act involved. Also, one of ordinary skill in the art will recognize that additional blocks that describe the processing may be added.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising.” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise. “Approximately” as applied to a particular value of a range applies to both values, and unless otherwise dependent on the precision of the instrument measuring the value, may indicate +/−10% of the stated value(s).

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

What is claimed is:

1. A system for detecting lesions on a multiparametric prostate using magnetic resonance imaging, comprising:
a magnetic resonance imaging (MRI) device for generating a plurality of MRI images of a prostate for a patient; and
at least one computing device in operable communication with the MRI device, the at least one computing device configured to detect lesions on the prostate of the patient by:
segmenting a plurality of MRI images to define:
a first zone of the prostate depicted in each of the plurality of images, and a second zone of the prostate depicted in each of the plurality of image, the second zone surrounding the first zone,
wherein each of the first zone and the second zone including anatomical data relating to the prostate;
collapsing the plurality of MRI images into a single, combined image;
rewindowing of the single, combined image by:
determining a first image intensity spectrum for the first zone of the prostate depicted in the single, combined image,
determining a second image intensity spectrum for the second zone of the prostate depicted in the single, combined image, and
defining:
a first image intensity threshold for the first zone of the prostate based on the first image intensity spectrum, and
a second image intensity threshold for the second zone of the prostate based on the second image intensity spectrum;
denoising of the single, combined image by:
dividing the single, combined image into a plurality of distinct regions, each of the plurality of regions associated with one of the first zone or the second zone of the prostate,
concatenating each of the plurality of regions to identify individual region image intensity values, comparing the region image intensity value for each of the plurality of regions to the first image intensity threshold or the second image intensity threshold based upon the region's association with the first zone or the second zone of the prostate, and identifying at least one positive region of the plurality of regions where the region image intensity values for the at least one positive region is equal to or greater than the first image intensity threshold or the second image intensity threshold; and
identifying a portion of the prostate including a detected lesion based on the identified at least one positive region of the plurality of regions, the identified portion of the prostate corresponding to:
the first zone or the second zone, and
an anatomic location of the prostate based on the anatomical data relating to the prostate.

2. The system of claim 1, wherein the at least one computing device is configured to detect the lesions on the prostate of the patient further by:
generating a report based on the identifying of the portion of the prostate including the detected lesion, the generated report including at least one of:
visual data relating to the identified portion of the prostate including the detected lesion,
probabilities for respective risk categories associated with the detected lesion, or options for modifications for the report.

3. The system of claim 32, wherein the generated report further includes a set of anatomic coordinates corresponding to the location of the detected lesion on the prostate, the set of anatomic coordinates based on the anatomical data relating to the prostate.

4. The system of claim 1, wherein the segmenting of the plurality of MRI images further includes: adjusting at least one of the first zone of the prostate or the second zone of the prostate based on user input.

5. The system of claim 1, wherein the plurality of MRI images include:
a low intensity MRI-image ranging from 0.7 T-1.2 T,
a mid-intensity MRI-image ranging from 1.3 T-1.9 T; and
a high-intensity MRI-image ranging from 2.0 T-3.0 T.

6. The system of claim 5, wherein the collapsing of the plurality of MRI images further includes:
aligning each of the low intensity MRI-image, the mid-intensity MRI image, and the high-intensity MRI-image based on the defined first zone and the second zone in each of the low intensity MRI-image, the mid-intensity MRI image, and the high-intensity MRI-image;
normalizing each of the aligned low intensity MRI-image, the mid-intensity MRI image, and the high-intensity MRI-image;
inverting a high-b value for each of the aligned low intensity MRI-image, the mid-intensity MRI image, and the high-intensity MRI-image;
segmenting the aligned low intensity MRI-image, the mid-intensity MRI image, and the high-intensity MRI-image; and
inverting the segmented and aligned low intensity MRI-image, the mid-intensity MRI image, and the high-intensity MRI-image.

7. The system of claim 1, wherein each of the plurality of distinct regions include a predetermined dimension.

8. The system of claim 1, wherein a prostate-specific antigen test determines the threshold value of a threshold tuning.

9. The system of claim 1, wherein the denoising of the single, combined image further includes:
in response to determining no region image intensity value for each of the plurality of regions is equal to or greater than the first image intensity threshold or the second image intensity threshold, adjusting at least one of:
the first image intensity threshold for the first zone of the prostate based on the first image intensity spectrum, or
the second image intensity threshold for the second zone of the prostate based on the second image intensity spectrum; and
comparing the region image intensity value for each of the plurality of regions to the adjusted first image intensity threshold or the adjusted second image intensity threshold.

10. A method of detecting lesions on a multiparametric prostate using magnetic resonance imaging (MRI), comprising:
segmenting a plurality of MRI images to define:
a first zone of the prostate depicted in each of the plurality of images, and a second zone of the prostate depicted in each of the plurality of image, the second zone surrounding the first zone,
wherein each of the first zone and the second zone including anatomical data relating to the prostate;
collapsing the plurality of MRI images into a single, combined image;
rewindowing of the single, combined image by:
determining a first image intensity spectrum for the first zone of the prostate depicted in the single, combined image,
determining a second image intensity spectrum for the second zone of the prostate depicted in the single, combined image, and
defining:
a first image intensity threshold for the first zone of the prostate based on the first image intensity spectrum, and
a second image intensity threshold for the second zone of the prostate based on the second image intensity spectrum;
denoising of the single, combined image by:
dividing the single, combined image into a plurality of distinct regions, each of the plurality of regions associated with one of the first zone or the second zone of the prostate,
concatenating each of the plurality of regions to identify individual region image intensity values,
comparing the region image intensity value for each of the plurality of regions to the first image intensity threshold or the second image intensity threshold based upon the region's association with the first zone or the second zone of the prostate, and
identifying at least one positive region of the plurality of regions where the region image intensity values for the at least one positive region is equal to or greater than the first image intensity threshold or the second image intensity threshold; and
identifying a portion of the prostate including a detected lesion based on the identified at least one positive region of the plurality of regions, the identified portion of the prostate corresponding to:
the first zone or the second zone, and
an anatomic location of the prostate based on the anatomical data relating to the prostate.

11. The method of claim 10, further comprising:
generating a report based on the identifying of the portion of the prostate including the detected lesion, the generated report including at least one of:
visual data relating to the identified portion of the prostate including the detected lesion,
probabilities for respective risk categories associated with the detected lesion, or options for modifications for the report.

12. The method of claim 11, wherein generating a report further includes generating a set of anatomic coordinates corresponding to the location of the detected lesion on the prostate, the set of anatomic coordinates based on the anatomical data relating to the prostate.

13. The method of claim 10, wherein segmenting of the plurality of MRI images further includes: adjusting at least one of the first zone of the prostate or the second zone of the prostate based on user input.

14. The method of claim 10, wherein collapsing of the plurality of MRI images further includes:
aligning each of the low intensity MRI-image, the mid-intensity MRI image, and the high-intensity MRI-image based on the defined first zone and the second zone in each of the low intensity MRI-image, the mid-intensity MRI image, and the high-intensity MRI-image;
normalizing each of the aligned low intensity MRI-image, the mid-intensity MRI image, and the high-intensity MRI-image;
inverting a high-b value for each of the aligned low intensity MRI-image, the mid-intensity MRI image, and the high-intensity MRI-image;
segmenting the aligned low intensity MRI-image, the mid-intensity MRI image, and the high-intensity MRI-image; and
inverting the segmented and aligned low intensity MRI-image, the mid-intensity MRI image, and the high-intensity MRI-image.

15. The method of claim 10, further including predetermining dimension for each of the plurality of distinct regions.

16. The method of claim 10, wherein determining the threshold value of a threshold tuning is based upon a prostate-specific antigen test.

17. The method of claim 10, wherein the denoising of the single, combined image further includes:
in response to determining no region image intensity value for each of the plurality of regions is equal to or greater than the first image intensity threshold or the second image intensity threshold, adjusting at least one of:
the first image intensity threshold for the first zone of the prostate based on the first image intensity spectrum, or
the second image intensity threshold for the second zone of the prostate based on the second image intensity spectrum; and
comparing the region image intensity value for each of the plurality of regions to the adjusted first image intensity threshold or the adjusted second image intensity threshold.

18. A computer program product for detecting lesions on a multiparametric prostate using magnetic resonance imaging (MRI), comprising a non-transitory computer readable storage medium having program instructions stored therein, the program instructions executable by a processor to cause a computing device to:
segment a plurality of MRI images to define:
a first zone of the prostate depicted in each of the plurality of images, and a second zone of the prostate depicted in each of the plurality of image, the second zone surrounding the first zone,
wherein each of the first zone and the second zone including anatomical data relating to the prostate;
collapse the plurality of MRI images into a single, combined image;
rewindow the single, combined image by:
determining a first image intensity spectrum for the first zone of the prostate depicted in the single, combined image,
determining a second image intensity spectrum for the second zone of the prostate depicted in the single, combined image, and
defining:
a first image intensity threshold for the first zone of the prostate based on the first image intensity spectrum, and
a second image intensity threshold for the second zone of the prostate based on the second image intensity spectrum;
denoise the single, combined image by:
dividing the single, combined image into a plurality of distinct regions, each of the plurality of regions associated with one of the first zone or the second zone of the prostate,
concatenating each of the plurality of regions to identify individual region I image intensity values,
comparing the region image intensity value for each of the plurality of regions to the first image intensity threshold or the second image intensity threshold based upon the region's association with the first zone or the second zone of the prostate, and
identifying at least one positive region of the plurality of regions where the region image intensity values for the at least one positive region is equal to or greater than the first image intensity threshold or the second image intensity threshold; and
identify a portion of the prostate including a detected lesion based on the identified at least one positive region of the plurality of regions, the identified portion of the prostate corresponding to:
the first zone or the second zone, and
an anatomic location of the prostate based on the anatomical data relating to the prostate.

19. The computer program product of claim 18, wherein the program instructions executable by the processor causes the computing device to further:
generate a report based on the identifying of the portion of the prostate including the detected lesion, the generated report including at least one of:
visual data relating to the identified portion of the prostate including the detected lesion,
probabilities for respective risk categories associated with the detected lesion, or options for modifications for the report.
